(19) United States
(12) Patent Application Publication
(10) Pub. No.: US 2007/0255065 A1
(43) Pub. Date: Nov. 1, 2007

(54) Benzodioxane and benzodioxolane derivatives and uses thereof

(75) Inventors: Qing Yu, Laval (CA); Michel Bernatchez, Montreal (CA); Rocco J. Galante, Oakland, NJ (US)

Correspondence Address: CHOATE, HALL & STEWART LLP/WYETH, PATENT GROUP, TWO INTERNATIONAL PLACE, BOSTON, MA, 02110, US

(73) Assignee: Wyeth, Madison, NJ (US)

(21) Appl. No.: 11/787,929

(22) Filed: Apr. 18, 2007

Related U.S. Application Data
(60) Provisional application No. 60/792,830, filed on Apr. 18, 2006.
(60) Provisional application No. 60/854,383, filed on Oct. 25, 2006.

(51) Int. Cl. C07D 319/14

(52) U.S. Cl. 549/362

(57) ABSTRACT

The present invention relates to methods for synthesizing compounds of formula I or pharmaceutically acceptable salts thereof: 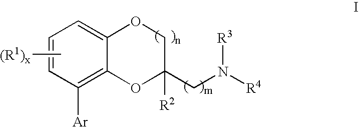
wherein each of R¹, R², R³, R⁴, x, m, n, and Ar are as defined, and described in classes and subclasses herein, which are agonists or partial agonists of the 2C subtype of brain serotonin receptors.

### CROSS-REFERENCE TO RELATED APPLICATIONS

The present invention claims priority to U.S. provisional patent application Ser. No. 60/792,830, filed Apr. 18, 2006, and U.S. provisional patent application Ser. No. 60/854,383, filed Oct. 25, 2006, the entirety of each of which is hereby incorporated herein by reference.

### FIELD OF THE INVENTION

The present invention relates to methods for synthesizing compounds useful as 5HT₂C agonists or partial agonists, derivatives thereof, and to intermediates thereto.

### BACKGROUND OF THE INVENTION

Schizophrenia affects approximately 5 million people. The most prevalent treatments for schizophrenia are currently the ‘atypical’ antipsychotics, which combine dopamine (D₂) and serotonin (5-HT_{2A}) receptor antagonism. Despite the reported improvements in efficacy and side-effect liability of atypical antipsychotics relative to typical antipsychotics, these compounds do not appear to adequately treat all the symptoms of schizophrenia and are accompanied by problematic side effects, such as weight gain (Allison, D. B., et al., Am. J. Psychiatry, 156: 1686-1696, 1999; Masand, P. S., Exp. Opin. Pharmacother. I: 377-389, 2000; Whitaker, R., Spectrum Life Sciences. Decision Resources. 2:1-9, 2000).

Atypical antipsychotics also bind with high affinity to 5-HT_{2C} receptors and function as 5-HT_{2C} receptor antagonists or inverse agonists. Weight gain is a problematic side effect associated with atypical antipsychotics such as clozapine and olanzapine, and it has been suggested that 5-HT_{2C} antagonism is responsible for the increased weight gain. Conversely, stimulation of the 5-HT_{2C} receptor is known to result in decreased food intake and body weight (Walsh et al., Psychopharmacology 124: 57-73, 1996; Cowen, P. J., et al., Human Psychopharmacology 10: 385-391, 1995; Rosenzweig-Lipson, S., et al., ASPET abstract, 2000).

Several lines of evidence support a role for 5-HT_{2C} receptor agonism or partial agonism as a treatment for schizophrenia. Studies suggest that 5-HT_{2C} antagonists increase synaptic levels of dopamine and may be effective in animal models of Parkinson's disease (Di Matteo, V., et al., Neuropharmacology 37: 265-272, 1998; Fox, S. H., et al., Experimental Neurology 151: 3549, 1998). Since the positive symptoms of schizophrenia are associated with increased levels of dopamine, compounds with actions opposite to those of 5-HT₂C antagonists, such as 5-HT_{2C} agonists and partial agonists, should reduce levels of synaptic dopamine. Recent studies have demonstrated that 5-HT_{2C} agonists decrease levels of dopamine in the prefrontal cortex and nucleus accumbens (Millan, M. J., et al., Neuropharmacology 37: 953-955, 1998; Di Matteo, V., et al., Neuropharmacology 38: 1195-1205, 1999; Di Giovanni, G., et al., Synapse 35: 5361, 2000), brain regions that are thought to mediate critical antipsychotic effects of drugs like clozapine. However, 5-HT_{2C} agonists do not decrease dopamine levels in the striatum, the brain region most closely associated with extrapyramidal side effects. In addition, a recent study demonstrates that 5-HT_{2C} agonists decrease firing in the ventral tegmental area (VTA), but not in the substantia nigra. The differential effects of 5-HT_{2C} agonists in the mesolimbic pathway relative to the nigrostriatal pathway suggest that 5-HT_{2C} agonists have limbic selectivity, and will be less likely to produce extrapyramidal side effects associated with typical antipsychotics.

### SUMMARY OF THE INVENTION

As described herein, the present invention provides methods for preparing compounds having activity as 5HT_{2C} agonists or partial agonists. These compounds are useful for treating schizophrenia, schizophreniform disorder, schizoaffective disorder, delusional disorder, substance-induced psychotic disorder, L-DOPA-induced psychosis, psychosis associated with Alzheimer's dementia, psychosis associated with Parkinson's disease, psychosis associated with Lewy body disease, dementia, memory deficit, intellectual deficit associated with Alzheimer's disease, bipolar disorders, depressive disorders, mood episodes, anxiety disorders, adjustment disorders, eating disorders, epilepsy, sleep disorders, migraines, sexual dysfunction, gastrointestinal disorders, obesity and its comorbidities, or a central nervous system deficiency associated with trauma, stroke, or spinal cord injury. Such compounds include those of formula I: 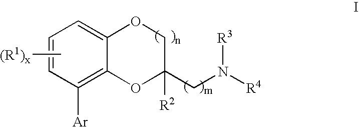
or a pharmaceutically acceptable salt thereof, wherein:
m is 1 or 2;
n is 0 or 1;
Ar is phenyl, an 8-10-membered bicyclic partially unsaturated or aryl ring, a 5-6 membered monocyclic heteroaryl having 1-4 heteroatoms independently selected from nitrogen, oxygen, or sulfur, or an 8-10-membered bicyclic partially unsaturated or heteroaryl ring having 1-5 heteroatoms independently selected from nitrogen, oxygen, or sulfur, wherein Ar is optionally substituted with one or more R^{x} groups;
each R^{x} is independently selected from —R, -Ph, —CN, halogen, —OR, —C(O)NH₂, —C(O)OR, —NHC(O)R, —SO₂R, or —NHSO₂R;
x is 0-3;
each R¹ is independently —R, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;

R² is hydrogen, C₁₋₃ alkyl, or —O(C₁₋₃ alkyl); and
each of R³ and R⁴ is independently hydrogen or C₁₋₆ aliphatic.

The present invention also provides synthetic intermediates useful for preparing such compounds.

### DETAILED DESCRIPTION OF CERTAIN EMBODIMENTS

The methods and intermediates of the present invention are useful for preparing compounds as described in U.S. provisional patent application Ser. No. 60/673,884, filed Apr. 22, 2005, the entirety of which is hereby incorporated herein by reference. In certain embodiments, the present compounds are generally prepared according to Scheme I set forth below:

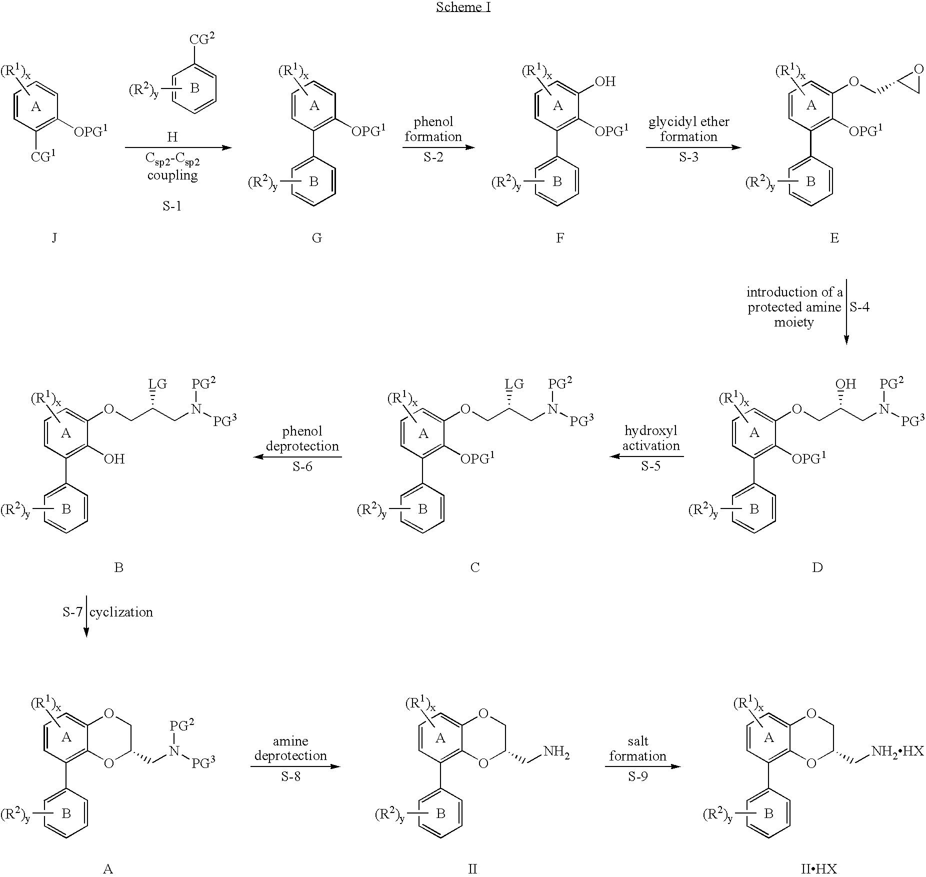

In Scheme I above, each of R¹, R², x, y, PG¹, PG², PG³, CG¹, CG², and LG is as defined below and in classes and subclasses as described herein.

In one aspect, the present invention provides methods for preparing chiral 2,8-disubstituted benzodioxane compounds of formulae A, II, and II•HX in enantiomerically enriched form according to the steps depicted in Scheme I, above.

At step S-1, a compound of formula J is coupled to a compound of formula H, via a Cₛₚ₂-Cₛₚ₂ coupling reaction between the carbon centers bearing complementary coupling groups CG¹ and CG² to provide a compound of formula G. Suitable coupling reactions are well known to one of ordinary skill in the art and typically involve one of the coupling groups being an electron-withdrawing group (e.g., Cl, Br, I, OTf, etc.), such that the resulting polar carbon-CG bond is susceptible to oxidative addition by an electron-rich metal (e.g., a low-valent palladium or nickel species), and the complementary coupling group being an electropositive group (e.g., boronic acids, boronic esters, boranes, stannanes, silyl species, zinc species, aluminum species, magnesium species, zirconium species, etc.), such that the carbon which bears the electropositive coupling group is susceptible to transfer to other electropositive species (e.g., a Pd^{II-IV} species or a Ni^{II-IV} species). Exemplary reactions and coupling groups include those described in Metal-Catalyzed Cross-Coupling Reactions, A. de Meijere and F. Diederich, Eds., 2^{nd} Edition, John Wiley & Sons, 2004. In certain embodiments, CG¹ in compounds of formula J is a boronic acid moiety, a boronic ester moiety, or a borane moiety. In other embodiments, CG¹ in compounds of formula J is a boronic ester moiety. According to one aspect of the present invention, CG¹ in compounds of formula J is a boronic acid moiety. In certain embodiments, CG² in compounds of formula H is Br, I, or OTf. According to one aspect of the present invention, CG² in compounds of formula H is Br. In certain embodiments, the transformation is catalyzed by a palladium species. According to one aspect of the invention, the transformation is catalyzed by palladium tetrakis triphenylphosphine. In certain embodiments, the coupling reaction is run with dimethoxyethane as solvent. In other embodiments, the reaction is heated. According to another aspect of the present invention, the reaction is run in the presence of sodium hydroxide. According to one aspect of the invention, the reaction is heated at reflux. According to another aspect of the invention, the reaction is run in the presence of sodium hydroxide.

The PG¹ group of formulae J, G, F, E, D, and C is a suitable hydroxyl protecting group. Protected hydroxyl groups (corresponding to OPG¹ of formulae J, G, F, E, D, and C) are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitably protected hydroxyl groups further include, but are not limited to, esters, carbonates, sulfonates allyl ethers, ethers, silyl ethers, alkyl ethers, arylalkyl ethers, and alkoxyalkyl ethers. Examples of suitable esters include formates, acetates, proprionates, pentanoates, crotonates, and benzoates. Specific examples of suitable esters include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benylbenzoate, 2,4,6trimethylbenzoate. Examples of suitable carbonates include 9-fluorenylmethyl, ethyl, 2,2,2-trichloroethyl, 2-trimethylsilyl)ethyl, 2-(phenylsulfonyl)ethyl, vinyl, allyl, and p-nitrobenzyl carbonate. Examples of suitable silyl ethers include trimethylsilyl, triethylsilyl, t-butyldimethylsilyl, t-butyldiphenylsilyl, triisopropylsilyl ether, and other trialkylsilyl ethers. Examples of suitable alkyl ethers include methyl, benzyl, p-methoxybenzyl, 3,4-dimethoxybenzyl, trityl, t-butyl, and allyl ether, or derivatives thereof. Alkoxyalkyl ethers include acetals such as methoxymethyl, methylthiomethyl, (2-methoxyethoxy)methyl, benzyloxymethyl, betadtrimethylsilyl)ethoxymethyl, and tetrahydropyran-2-yl ether. Examples of suitable arylalkyl ethers include benzyl, p-methoxybenzyl (MPM), 3,4-dimethoxybenzyl, 0-nitrobenzyl, p-nitrobenzyl, p-halobenzyl, 2,6-dichlorobenzyl, p-cyanobenzyl, 2- and 4-picolyl ethers. According to one aspect of the present invention, the PG¹ group of formulae J, G, F, E, D, and C is methyl.

Each R¹ group of formulae J, G, F, E, D, C, B, A, II, and II•HX is independently —R, -Ph, —CN, halogen, —OR, —C(O)NH₂, —(O)OR, —NHC(O)R, —SO₂R, or —NHSO₂R, wherein each R is independently hydrogen, C₁₋₆ aliphatic or C₁₋₆ fluoroaliphatic. Examples of suitable R¹ groups include methyl, ethyl, isopropyl, chloro, fluoro, and bromo. According to one aspect of the present invention, R¹ is fluoro. According to another aspect of the present invention, R¹ in ring A of compounds of formulae J, G, F, E, D, C, B, A, II, and II•HX, is located at the ring position that corresponds to the position para to OPG¹ in formula J.

The numeral x of formulae J, G, F, E, D, C, B, A, II, and II•HX is 0-3. According to one aspect of the present invention, x is 1.

Each R² group of formulae H, G, F, E, D, C, B, A, II, and II•HX is independently R, -Ph, —CN, halogen, —OR, —C(O)NH₂, —C(O)OR, —NHC(O)R, —SO₂R, or —NHSO₂R, wherein each R is independently hydrogen, C₁₋₆ aliphatic or C1-6 fluoroaliphatic. Examples of suitable R² groups include methyl, ethyl, isopropyl, chloro, fluoro, bromo, methoxyl, trifluoromethyl, phenyl, cyano, ethoxyl, trifluoromethoxyl, and isopropoxyl. According to one aspect of the present invention, R² is chloro. According to another aspect of the present invention, at least one R² in ring B of compounds of formulae H, G, F, E, D, C, B, A, II, and II•HX, is located at one of the two ring positions that correspond to the positions ortho to CG² in formula H. According to yet another aspect of the present invention, an R² group is located at each of the two ring positions that correspond to the positions ortho to CG² in formula H. In certain embodiments, ring B is selected from those moieties depicted in Table 1, below, wherein the [symbol] represents the point of attachment of ring B to CG² in compounds of formula H, or the point of attachment of ring B to ring A in compounds of formulae G, F, E, D, C, B, A, II, and II•IIX.

The numeral y of formulae H, G, F, E, D, C, B, A, II, and II•HX is 0-5. According to one aspect of the invention, y is 2.

TABLE 1

| | |
|---|---|
| 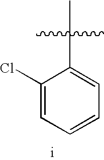 | 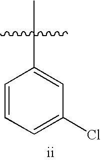 |
| 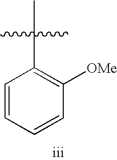 | 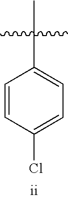 |
| 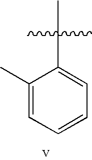 | 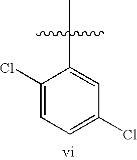 |
| 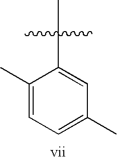 | 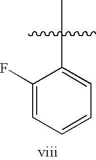 |
| 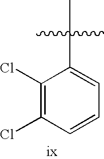 | 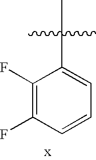 |
| 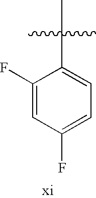 | 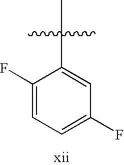 |
| 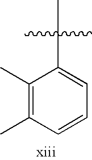 | 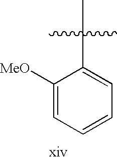 |
| 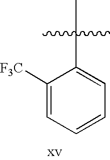 | 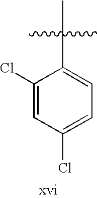 |
| 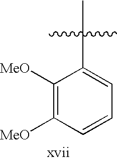 | 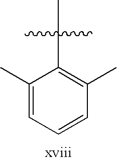 |
| 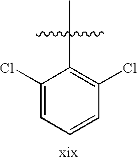 | 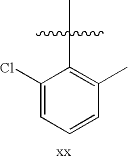 |
| 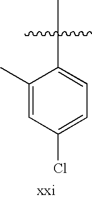 | 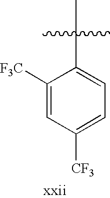 |
| 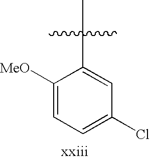 | 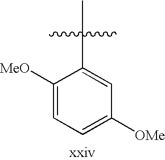 |
| 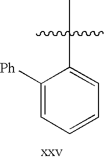 | 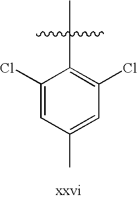 |
| 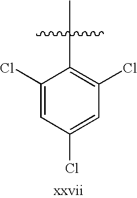 | 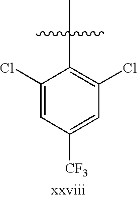 |
| 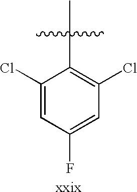 | 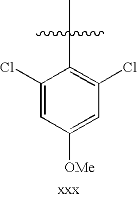 |
| 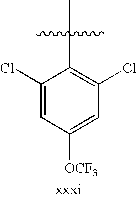 | 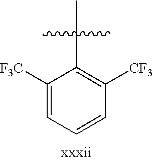 |
| 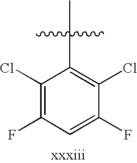 | 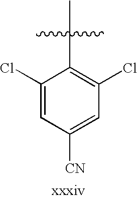 |
| 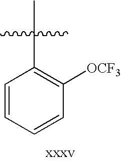 | 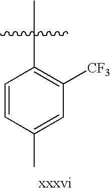 |
| 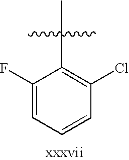 | 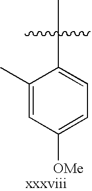 |
| 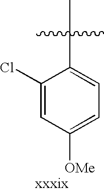 | 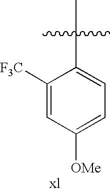 |
| 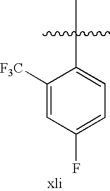 | 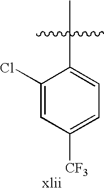 |
| 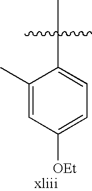 | 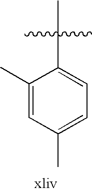 |
| 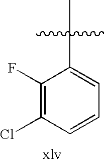 | 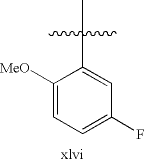 |
| 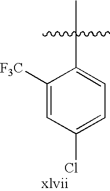 | 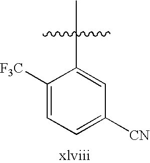 |
| 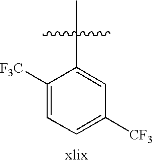 | 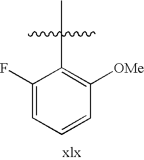 |
| 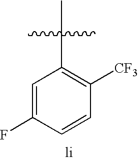 | 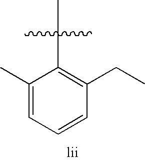 |
| 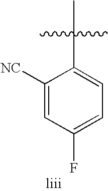 | 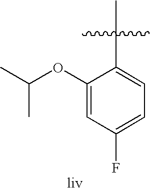 |
| 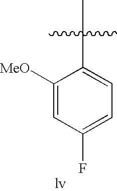 | |

At step S-2, a hydroxyl group is introduced at the open ortho position relative to the OPG¹ group of formula G. One of ordinary skill in the art will recognize that there are a wide variety of reactions and reaction sequences that can be employed to accomplish this transformation; see generally, March's Advanced Organic Chemistry: Reactions, Mechanisms, and Structure, M. B. Smith and J. March, 5^{th} Edition, John Wiley & Sons, 2001 and Comprehensive Organic Transformaions, R. C. Larock, 2^{nd} Edition, John Wiley & Sons, 1999. Exemplary sequences include initial directed orthometallation followed by either (a) direct treatment with an electrophilic oxygen source; (b) treatment with a borate ester followed by oxidative workup of the resulting boronic ester or acid; or (c) treatment with a reagent that will allow the introduction of a formyl group (e.g., methyl formate, dimethylformamide) followed by subsequent Baeyer-Villiger reaction; for the above methods, see, e.g., Snieckus, V. Chem. Rev. 1990, 90, 879 and Schlosser, M. Angew. Chem. Int. Ed. 2005, 44, 376. Alternatively, direct orthoformylation may be utilized, followed by a Baeyer-Villiger reaction; see, e.g., Laird, T. in Comprehensive Organic Chemistry, Stoddart, J. F., Ed., Pergamon, Oxford 1979, Vol. 1, p 1105 and Hofsløkken, N. U.; Skattebøl, L. Acta Chem. Scand. 1999, 53, 258.

Another exemplary method for introducing a hydroxyl group at step S-2 involves halogenation followed by a metallation/transmetallation sequence to afford a boronic acid, boronic ester, or borane, followed by peroxide oxidation; see, generally, de Meijere (2004) and Snieckus (1990).

According to one aspect of the present invention, a compound of formula G is first brominated, then is subjected to halogen-metal exchange to afford an intermediate arylmetal compound that is allowed to react with a borate ester to afford, following aqueous workup, a boronic acid, which is subsequently oxidized to provide a phenol of formula F, as depicted in Scheme II below. According to another aspect of the invention, the brominating agent is N-bromosuccinimide. In certain embodiments, the bromination is conducted in the presence of para-toluenesulfonic acid and acetic acid.

According to yet another aspect of the invention, the metallation/transmetallation sequence involves initial magnesium-halogen exchange, followed by treatment with a trialkyl borate. In certain embodiments, the magnesium-halogen exchange is accomplished by treating the intermediate aryl bromide with isopropylmagnesium bromide. According to one aspect of the invention, the magnesium-halogen exchange is conducted in tetrahydrofuran (THF). In other embodiments, the trialkyl borate is triisopropylborate [B(OiPr)₃]. In certain embodiments, the metallation/transmetallation step is conducted at a temperature that is between about −20° C. and about 20° C. In other embodiments, the boronic acid is oxidized with hydrogen peroxide (H₂O₂) to afford compounds of formula F. In other embodiments, the boronic acid is oxidized with peroxyacetic acid (also called peracetic acid) or meta-chloroperoxybenzoic acid (mCPBA). One of ordinary skill in the art will recognize that such procedures for magnesium-halogen exchange followed by transmetallation to a boron-containing moiety, followed by oxidation to the phenol can be performed without isolation of the respective intermediate species.

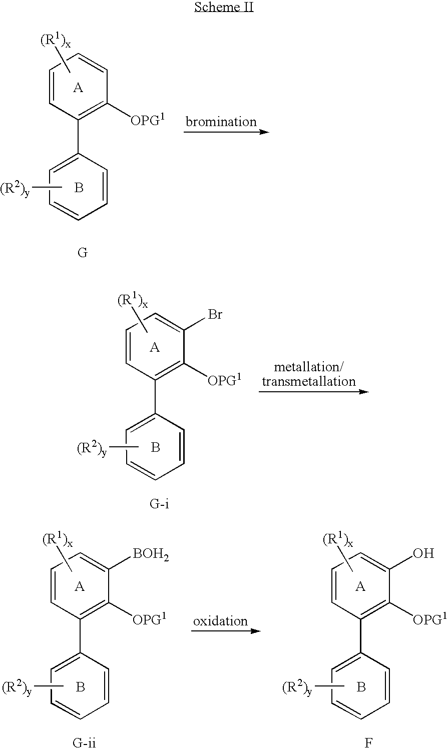

At step S-3, a compound of formula F is glycidated on the phenol oxygen. Exemplary reagents that may be used to promote glycidation include epichlorohydrin, epibromohydrin, oxiranylmethyl p-toluenesulfonate (also called: oxiranylmethyl tosylate or glycidyl tosylate), oxiranylmethyl methanesulfonate (oxiranylmethyl mesylate or glycidyl mesylate), and oxiranylmethyl trifluoromethanesulfonate (oxiranylmethyl triflate or glycidyl triflate). According to one aspect of the present invention, the activated glycidol equivalent is gycidyl tosylate.

In certain embodiments, at step S-3, a compound of formula F is treated with a base to form the corresponding metal phenoxide salt, which is then allowed to react with an activated glycidol equivalent to afford a compound of formula E. In other embodiments, the base employed is selected from sodium hydroxide (NaOH), potassium carbonate (K₂CO₃), potassium tert-butoxide (KOtBu), lithium diisopropylamide (LDA), lithium hexamethyldisilazide (LHMDS), or sodium hydride (NaH). According to one aspect of the present invention, the base is potassium tert-butoxide.

In certain embodiments, the reaction is conducted in the presence of a polar aprotic solvent. Exemplary polar aprotic solvents include dimethylformamide (DMF), N-methylpyrrolidine (NMP), dimethylacetamide (DMA), dioxane, tetrahydrofuran (THF), and dimethylsulfoxide (DMSO). In certain embodiments, the reaction is conducted using dimethylformamide (DMF), N-methylpyrrolidine (NMP), or dimethylacetamide (DMA) as solvent. In other embodiments, DMF is employed as solvent. In certain embodiments, the reaction is heated. In other embodiments, the reaction is conducted at a temperature that is between about 20° C. and about 100° C.

One of ordinary skill in the art will recognize that the activated glycidol equivalents contain a stereogenic carbon, and accordingly, compounds of formula E contain a stereogenic carbon corresponding thereto.

In certain embodiments, the glycidol equivalent employed at step S-3 is enantiomerically enriched, and accordingly, the mixture of enantiomers of formula E that are generated in this step is enriched in one of the enantiomers. While a single stereochemical isomer is depicted for formulae E, D, C, B, A, II, and II•HX in Scheme I, it will be appreciated that mixtures of enantiomers of these formulae are accessible enriched in either enantiomer via the present invention. As used herein, the terms “enantiomerically enriched” and “enantioenriched” denote that one enantiomer makes up at least 75% of the preparation. In certain embodiments, the terms denote that one enantiomer makes up at least 80% of the preparation. In other embodiments, the terms denote that at least 90% of the preparation is one of the enantiomers. In other embodiments, the terms denote that at least 95% of the preparation is one of the enantiomers. In still other embodiments, the terms denote that at least 97.5% of the preparation is one of the enantiomers. In yet other embodiments, the terms denote that at least 99% of the preparation is one of the enantiomers. In still other embodiments, the terms denote that at least 99.5% of the preparation is one of the enantiomers. In yet another embodiment, the terms denote that the preparation consists of a single enantiomer to the limits of detection (also referred to as “enantiopure”). As used herein, when “enantioenriched” or “enantiomerically enriched” are used to describe a singular noun (e.g., “an enantioenriched compound of formula II” or “an enantioenriched chiral acid”), it should be understood that the “compound” or “acid” may be enantiopure, or may in fact be an enantioenriched mixture of enantiomers. Similarly, when “racemic” is used to describe a singular noun (e.g., “a racemic compound of formula E”), it should be understood that the term is in fact describing a 1:1 mixture of enantiomers.

At step S-4, a protected amine moiety is introduced via epoxide-opening to afford compounds of formula D. In compounds of formulae D, C, B, and A, PG² and PG³ are amino protecting groups. Protected amines are well known in the art and include those described in detail in Greene (1999). Suitable mono-protected amines further include, but are not limited to, aralkylamines, carbamates, allyl amines, amides, and the like. Examples of suitable mono-protected amino moieties include t-butyloxycarbonylamino (—NHBOC), ethyloxycarbonylamino, methyloxycarbonylamino, trichloroethyloxycarbonylamino, allyloxycarbonylamino (-NHAlloc), benzyloxocarbonylamino (-NHCBZ), allylamino, benzylamino (-NHBn), fluorenylmethylcarbonyl (-NHFmoc), formamido, acetamido, chloroacetamido, dichloroacetamido, trichloroacetamido, phenylacetamido, trifluoroacetamido, benzamido, t-butyldiphenylsilyl, and the like. Suitable di-protected amines include amines that are substituted with two substituents independently selected from those described above as mono-protected amines, and further include cyclic imides, such as phthalimide, maleimide, succinimide, and the like. Suitable di-protected amines also include pyrroles and the like, and 2,2,5,5-tetramethyl-[1,2,5]azadisilolidine and the like. Notwithstanding the definition above, one of either PG² or PG³ in compounds of formulae D, C, B, and A may be hydrogen. Also notwithstanding the definitions above, the —N(PG²)(PG³) moiety of formulae D, C, B, and A may be azido. According to one aspect of the invention, the —N(PG²)(PG³) moiety of formulae D, C, B, and A, is phthalimido. According to another aspect of the invention, at step S-4, a compound of formula E is treated with potassium phthalimide to generate compounds of formula D in which the —N(PG²)(PG³) moiety is phthalimido.

In certain embodiments, step S-4 is performed with heating. In other embodiments, the reaction is conducted at a temperature that is between about 40° C. and about 110° C. In other embodiments, the reaction is run at about 80° C.

In certain embodiments, step S-4 is conducted in the presence of a polar aprotic solvent. Exemplary polar aprotic solvents include dimethylformamide (DMF), N-methylpyrrolidine (NMP), dimethylacetamide (DMA), dioxane, tetrahydrofuran (THF), and dimethylsulfoxide (DMSO). In certain embodiments, the reaction is conducted in dimethylformamide (DMF), N-methylpyrrolidine (NMP), or dimethylacetamide (DMA). In other embodiments, the reaction is conducted in DMF.

In certain embodiments, steps S-3 and S-4 may be conducted without isolating compounds of formula E. Accordingly, one aspect of the present invention is a procedure of glycidation followed by epoxide-opening to introduce a protected amine moiety without isolation of the intermediate glycidated species. In certain embodiments, the phthalimide is directly added to the reaction mixture in which the glycidated species was formed.

At step S-5, the hydroxyl group of compounds of formula D is activated such that it becomes leaving group LG that is subject to nucleophilic displacement. A suitable “leaving group” that is “subject to nucleophilic displacement” is a chemical group that is readily displaced by a desired incoming nucleophilic chemical entity. Suitable leaving groups are well known in the art, e.g., see, Smith and March (2001). Such leaving groups include, but are not limited to, halogen, alkoxy, sulphonyloxy, optionally substituted alkylsulphonyloxy, optionally substituted alkenylsulfonyloxy, optionally substituted arylsulfonyloxy, and diazonium moieties. For the above mentioned “optionally substituted” moieties, the moieties may be optionally substituted with C₁₋₄ aliphatic, fluoro-substituted C₁₋₄ aliphatic, halogen, or nitro. Examples of suitable leaving groups include chloro, iodo, bromo, fluoro, methanesulfonyloxy (mesyloxy), tosyloxy, triflyloxy, nitro-phenylsulfonyloxy (nosyloxy), and bromo-phenylsulfonyloxy (brosyloxy). According to one aspect of the present invention, LG in compounds of formula C is methanesulfonyloxy (mesyloxy). According to another aspect of the invention, a compound of formula D is allowed to react with methanesulfonyl chloride (mesyl chloride) to afford a compound of formula C in which LG is methanesulfonyloxy (mesyloxy).

In certain embodiments step S-5 is performed in ethereal solvents, ester solvents, halogenated hydrocarbon solvents, or nitrile solvents. In certain embodiments this reaction is performed in tetrahydrofuran (THF), dichloromethane, acetonitrile, or isopropyl acetate. In other embodiments the reaction is run in THF. According to one aspect of the present invention, the reaction is run in the presence of suitable base. Exemplary bases include tertiary amines such as triethylamine (TEA), pyridine, and DIPEA. In certain embodiments, the reaction is run at a temperature that is between about −20° C. and about 40° C. In other embodiments, the reaction is conducted at a temperature of about 0° C.

At step S-6, removal of the PG¹ protecting group in compounds of formula C affords the free phenol-containing compounds of formula B. Procedures for the removal of suitable hydroxyl protecting groups are well known in the art; see Green (1999). In certain embodiments, where PG¹ is methyl, PG¹ is removed by treatment of a compound of formula C with BBr₃, iodotrimethylsilane, or a combination of BCl₃ and LiI. According to one aspect of the present invention, where PG¹ is methyl, PG¹ is removed by treatment of a compound of formula C with BBr₃. In certain embodiments this step is conducted using toluene, dichloromethane, or isopropyl acetate as solvent. In other embodiments, this step is conducted using toluene as solvent. In certain embodiments, the reaction is conducted at a temperature between about −20° C. and about 40° C.

At step S-7, a compound of formula B is allowed to cyclize to afford a compound of formula A. One of ordinary skill in the art would recognize that a wide variety of reaction conditions are useful for promoting this reaction, therefore a wide variety of reaction conditions are contemplated. For example, the reaction may be conducted with or without thermal excitation, with or without base catalysis, and in protic or aprotic media. According to one aspect of the invention, the reaction is promoted by the addition of potassium carbonate, potassium t-butoxide, sodium hydride, lithium diisopropylamide, or lithium hexamethyldisilazide to a compound of formula B. According to another aspect of the invention, the reaction is promoted by the addition of potassium carbonate. In certain embodiments, the reaction is conducted with dimethylformamide, N-methylpyrollidone, or dimethylacetamide as solvent. In other embodiments, the reaction is conducted with dimethylformamide as solvent. In certain embodiments, the reaction is conducted at a temperature between about 10° C. and about 60° C.

At step S-8, removal of the PG² and PG³ protecting groups in compounds of formula A affords the free amine-containing compounds of formula II. Procedures for the removal of suitable amino protecting groups are well known in the art; see Green (1999). In certain embodiments, where the —N(PG²)(PG³) moiety of formulae A is phthalimido, PG² and PG³ are removed by treatment with a primary amine or other methods known in the art. In certain embodiments, the phthalimide group is removed with hydrazine or methylamine. In other embodiments, where the —N(PG²)(PG³) moiety of formulae A is phthalimido, PG² and PG³ are removed by treatment with hydrazine. In certain embodiments, this transformation is conducted with a mixture of water in one or more of ethanol, methanol, isopropanol, or tetrahydrofuran as solvent. In other embodiments, this transformation is conducted with ethanol as solvent. In certain embodiments, the reaction is conducted at a temperature between about 40° C. and about 90° C. In other embodiments the reaction is conducted with an ethanol-water mix as solvent at reflux.

One of ordinary skill in the art will appreciate that a compound of formula II, as prepared by the methods of the present invention, may be treated with a suitable Brønsted acid, HX, as depicted in step S-9, to form a salt thereof (represented by formula II•HX). Exemplary acids include hydrogen halides, carboxylic acids, sulfonic acids, sulfuric acid, and phosphoric acid. According to one aspect of the present invention, a compound of formula II is treated with HCl to form a compound of formula II•HX wherein X is Cl. In certain embodiments, where the acid is HCl, it is introduced into the medium containing the compound of formula II in gaseous form. In other embodiments, the acid is introduced into the medium containing the compound of formula II as a solution in methanol, ethanol, isopropanol, or water. In yet other embodiments, the acid is introduced into the medium containing the compound of formula II as a solution in isopropanol or TBME. In certain embodiments, the medium containing the compound of formula II is isopropanol.

One skilled in the art will appreciate that the enantiomeric excess of any of formulae E, D, C, B, A, II, and II•HX may be increased through a variety of means. Exemplary methods by which this may be accomplished include (a) the separation of enantiomers by chiral chromatographic methods, (b) selective crystallization of one enantiomer over the other, optionally by seeding a solution of the mixture of enantiomers with a crystal enriched in the desired enantiomer, (c) selective reaction of one enantiomer over the other with an enantioenriched chiral reaction partner, (d) selective reaction of one enantiomer over the other through chiral catalyst-promoted transformations (including enzymatic transformations), and (e) conversion of both enantiomers to corresponding diastereomers via either covalent or ionic bonding to a different enantiomerically enriched chiral species, followed by separation of the resulting diastereomers based upon their differing physical properties; for the above methods, see generally, Stereochemistry of Organic Compounds, E. L. Eliel and S. H. Silen, 1994; Enantiomers, Racemates and Resolutions, Jacques, et al. Wiley Interscience, New York, 1981; Wilen, S. H. et al., Tetrahedron 1977, 33, 2725; Tables of Resolving Agents and Optical Resolutions, Wilen, S. H. (E. L. Eliel, Ed.), Univ. of Notre Dame Press, Notre Dame, Ind. 1972. One of ordinary skill in the art will recognize that for preceding method (e), where both enantiomers of the compound of interest are converted by chemical means to a different chemical entity, that a subsequent step (or subsequent steps) may be necessary to reacquire the initial compounds.

In certain embodiments, a mixture of enantiomers of any of formulae E, D, C, B, A, II, and II•HX is subjected to one or more steps to increase the enantiomeric excess thereof. According to one aspect of the present invention, a mixture of enantiomers of formula A is dissolved in a suitable solvent and crystallized therefrom to afford a crystalline product that is further enriched in a single enantiomer. In certain embodiments, the suitable solvent is selected from toluene, ethyl acetate, dimethylformamide, and tetrahydrofuran. In other embodiments, the suitable solvent is toluene. In other embodiments, the mixture of enantiomers of formula A is dissolved in a suitable solvent at a temperature between about 70° C. and about 90° C. In certain embodiments, the crystallization occurs on cooling of a heated solution of enantiomers of formula A.

It will be appreciated that enantioenrichment of the crystals also results in enrichment of the opposite enantiomer of the mother liquor. Accordingly, it is contemplated that both enantiomers may be obtained in enriched form. Accordingly, in another aspect of the present invention, a mixture of enantiomers of formula A is dissolved in a suitable solvent and the enantiomer not desire is crystallized therefrom to afford a crystalline product that is further enriched in a single enantiomer and a mother liquor enriched in the desired enantiomer. In certain embodiments, the suitable solvent is selected from toluene, ethyl acetate, dimethylformamide, and tetrahydrofuran. In other embodiments, the suitable solvent is toluene. In other embodiments, the mixture of enantiomers of formula A is dissolved in a suitable solvent at a temperature between about 70° C. and about 90° C. In certain embodiments, the crystallization occurs on cooling of a heated solution of enantiomers of formula A and the mother liquor is collected to obtain the desired enantiomer in enriched form.

According to one aspect of the present invention, a mixture of enantiomers of formula II is allowed to react with an enantiomerically enriched chiral acid, and the diastereomeric excess of the resulting salts is increased by selective crystallization of one of the diastereomers over the others. According to yet another aspect of the present invention, the chiral acid employed in the aforementioned crystallizations is dibenzoyltartaric acid. In certain embodiments, the diastereomeric salts are formed by combining enantiomers of formula II with enantioenriched chiral acid in tetrahydrofuran, isopropanol, ethanol, water, or mixtures thereof, followed by optional heating to temperatures as high as the reflux temperature of the solvent used. In other embodiments, the aforementioned diastereomeric salts are formed in refluxing tetrahydrofuran. In certain embodiments, the crystallization of diastereomeric salts of compounds of formula II is from a solution in tetrahydrofuran, isopropanol, ethanol, water, or mixtures thereof. In other embodiments, said crystallization is from a solution in tetrahydrofuran. In yet other embodiments, the crystallization occurs on cooling of a heated solution of the salt. In certain embodiments, the solution is heated as high as the reflux temperature of the solvent and cooled to as low as 10° C. Compounds of formula II can be obtained from the diastereomeric salts by treatment with a suitable base in a suitable solvent. One of ordinary skill in the art will appreciate that a wide variety of bases and solvents are appropriate for this purpose, thus a large variety thereof is envisioned. In certain embodiments, the suitable base is sodium hydroxide and the suitable solvent is selected from water, tert-butyl methyl ether, or a mixture thereof. According to another aspect of the present invention, the above-mentioned selective crystallization procedures are optionally repeated to further increase the enantiomeric or diastereomeric excesses of the compounds that are being crystallized.

It is further recognized that atropisomers of the present compounds may exit. The present invention thus encompasses atropisomeric forms of compounds of formulae G, F, E, D, C, B, A, II, and II•HX as defined above, and in classes and subclasses described above and herein.

As used herein, the term “aliphatic” or “aliphatic group”, as used herein, means a straight-chain (i.e., unbranched) or branched, hydrocarbon chain that is completely saturated or that contains one or more units of unsaturation, or a monocyclic hydrocarbon that is completely saturated or that contains one or more units of unsaturation, but which is not aromatic (also referred to herein as “carbocycle” or “cycloaliphatic”), that has a single point of attachment to the rest of the molecule. In certain embodiments, aliphatic groups contain 1-6 carbon atoms, and in yet other embodiments, aliphatic groups contain 1-3 carbon atoms. In some embodiments, “cycloaliphatic” (or “carbocycle”) refers to a monocyclic C₃-C₆ hydrocarbon that is completely saturated or that contains one or more units of unsaturation, but which is not aromatic, that has a single point of attachment to the rest of the molecule. Such groups include cycloalkyl, cycloalkenyl, and cycloalkynyl groups. Suitable aliphatic groups include, but are not limited to, linear or branched, alkyl, alkenyl, alkynyl groups and hybrids thereof such as (cycloalkyl)alkyl, (cycloalkenyl)alkyl or (cycloalkyl)alkenyl.

The term “unsaturated,” as used herein, means that a moiety has one or more units of unsaturation.

The term “alkyl,” as used herein, refers to a hydrocarbon chain having up to 6 carbon atoms. The term “alkyl” includes, but is not limited to, straight and branched chains such as methyl, ethyl, n-propyl, isopropyl, n-butyl, isobutyl, sec-butyl, t-butyl, n-pentyl, iso-pentyl, 1-methyl-butyl, 2-methyl-butyl, n-hexyl, 1-methyl-pentyl, 2-methyl-pentyl, 3-methyl-pentyl, or 4methyl-pentyl.

The terms “halogen” or “halo,” as used herein, refer to a chloro (—Cl), bromo (—Br), fluoro (—F) or iodo (—I) atom.

The term “haloaliphatic,” as used herein, refers to an aliphatic group, as defined herein, that has one or more halogen substituents. In certain embodiment, every hydrogen atom on said aliphatic group is replaced by a halogen atom. Such haloaliphatic groups include —CF₃.

The term “fluoroaliphatic,” as used herein, an aliphatic group, as defined herein, that has one or more fluorine substituents. In a certain embodiment, a fluoroaliphatic group is a fluoroalkyl group.

The term “fluoroalkyl,” as used herein, refers to an alkyl group, as defined herein, that has one or more fluorine substituents. In certain embodiment, every hydrogen atom on said alkyl group is replaced by a fluorine atom.

The term “Ph,” as used herein, refers to a phenyl group.

The term “alkenyl,” as used herein refers to an aliphatic straight or branched hydrocarbon chain having 2 to 8 carbon atoms that may contain 1 to 3 double bonds. Examples of alkenyl groups include vinyl, prop-1-enyl, allyl, methallyl, but-1-enyl, but-2-enyl, but-3-enyl, or 3,3-dimethylbut-1-enyl. In some embodiments, the alkenyl is preferably a branched alkenyl of 3 to 8 carbon atoms.

The term “pharmaceutically acceptable salts” or “pharmaceutically acceptable salt” includes acid addition salts, that is salts derived from treating a compound of formula II with an organic or inorganic acid such as, for example, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, or similarly known acceptable acids. Where a compound of formula I contains a substituent with acidic properties, the term also includes salts derived from bases, for example, sodium salts. In certain embodiments, the present invention provides the hydrochloride salt of a compound of formula II.

According to another aspect, the present invention provides an alternate method for preparing a compound of formula A from a compound of formula E as depicted in Scheme III, below: 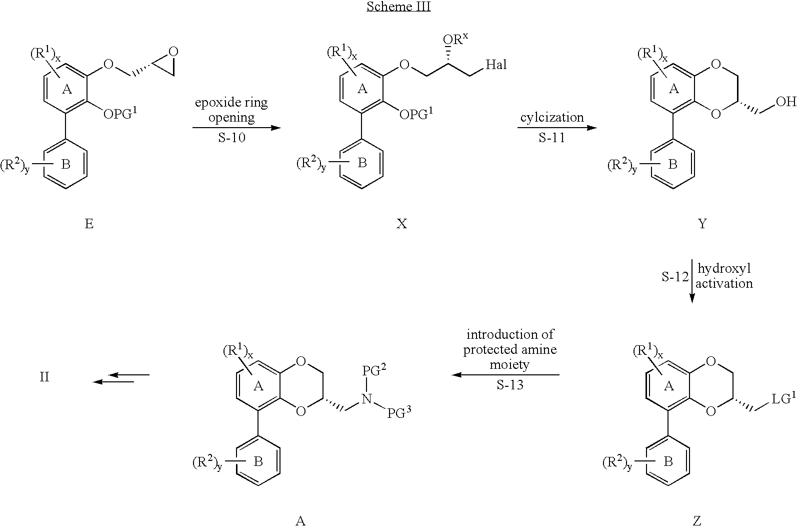
wherein each of R¹, R², x, y, PG¹, PG^{2,} and PG³ are as defined above, and in classes and subclasses described above and herein.

In step S-10, the epoxide ring of a compound of formula E is opened to form a compound of formula X, wherein R^{x} is hydrogen or acetyl, and Hal is a halogen. One of ordinary skill in the art will recognize that there are various methods known for the transformation depicted at step S-10. In certain embodiments, the ring opening step S-10 is performed by treating a compound of formula E with HBr in acetic acid.

At step S-11, a compound of formula X is allowed to cyclize to afford a compound of formula Y. One of ordinary skill in the art would recognize that a wide variety of reaction conditions are useful for promoting this reaction, therefore a wide variety of reaction conditions are contemplated. For example, the reaction may be conducted with or without thermal excitation, with or without base catalysis, and in protic or aprotic media. According to one aspect of the invention, the reaction is promoted by the addition of potassium carbonate, potassium t-butoxide, sodium hydride, lithium diisopropylamide, or lithium hexamethyldisilazide to a compound of formula X. According to another aspect of the invention, the reaction is promoted by the addition of sodium hydroxide. In certain embodiments, the reaction is conducted with an alcohol or a dipolar aprotic solvent, or mixtures thereof. In other embodiments, S-11 is performed in methanol, ethanol, dimethylformamide, N-methylpyrollidone, or dimethylacetamide, or mixtures thereof as solvent. In other embodiments, the reaction is conducted with methanol as solvent. In certain embodiments, the reaction is conducted at a temperature between about 0° C. and about 40° C.

At step S-12, the hydroxyl group of compounds of formula Y is activated to form compounds of formula Z wherein LG¹ is a suitable leaving group that is subject to nucleophilic displacement. A suitable “leaving group” that is “subject to nucleophilic displacement” is a chemical group that is readily displaced by a desired incoming nucleophilic chemical entity. Suitable leaving groups are well known in the art, e.g., see, Smith and March (2001). Such leaving groups include, but are not limited to, halogen, alkoxy, sulphonyloxy, optionally substituted alkylsulphonyloxy, optionally substituted alkenylsulfonyloxy, optionally substituted arylsulfonyloxy, and diazonium moieties. For the above mentioned “optionally substituted” moieties, the moieties may be optionally substituted with C₁₋₄ aliphatic, fluoro-substituted C₁₋₄ aliphatic, halogen, or nitro. Examples of suitable leaving groups include chloro, iodo, bromo, fluoro, methanesulfonyloxy (mesyloxy), tosyloxy, triflyloxy, nitro-phenylsulfonyloxy (nosyloxy), and bromo-phenylsulfonyloxy (brosyloxy). According to one aspect of the present invention, LG¹ in compounds of formula Z is toluenesulfonyloxy (tosyloxy). According to another aspect of the invention, a compound of formula Y is allowed to react with toluenesulfonyl chloride (tosyl chloride) to afford a compound of formula Z in which LG¹ is toluenesulfonyloxy (tosyloxy).

In certain embodiments step S-12 is performed in ethereal solvents, ester solvents, halogenated hydrocarbon solvents, or nitrile solvents. In certain embodiments this reaction is performed in tetrahydrofuran (THF), dichloromethane, acetonitrile, or isopropyl acetate. In other embodiments the reaction is run in dichloromethane. According to one aspect of the present invention, the reaction is run in the presence of suitable base. Exemplary bases include tertiary amines such as isopropylethylamine, triethylamine (TEA), pyridine, and DIPEA. Step S-12 is optionally performed in the presence of an additional base, such as dimethylaminopyridine (DMAP). In certain embodiments, the reaction is run at a temperature that is between about −20° C. and about 40° C. In other embodiments, the reaction is conducted at a temperature of about 0° C. or ambient temperature.

At step S-13, a protected amine moiety is introduced via displacement of the LG¹ group of formula Z to afford compounds of formula A, wherein PG² and PG³ are amino protecting groups. Protected amines are well known in the art and include those described in detail in Greene (1999). Suitable mono-protected amines further include, but are not limited to, aralkylamines, carbamates, allyl amines, amides, and the like. Examples of suitable mono-protected amino moieties include t-butyloxycarbonylamino (—NHBOC), ethyloxycarbonylamino, methyloxycarbonylamino, trichloroethyloxycarbonylamino, allyloxycarbonylamino (-NHAlloc), benzyloxocarbonylamino (-NHCBZ), allylamino, benzylamino (-NHBn), fluorenylmethylcarbonyl (-NHFmoc), formamido, acetamido, chloroacetamido, dichloroacetamido, trichloroacetamido, phenylacetamido, trifluoroacetamido, benzamido, t-butyldiphenylsilyl, and the like. Suitable di-protected amines include amines that are substituted with two substituents independently selected from those described above as mono-protected amines, and further include cyclic imides, such as phthalimide, maleimide, succinimide, and the like. Suitable di-protected amines also include pyrroles and the like, and 2,2,5,5-tetramethyl-[1,2,5]azadisilolidine and the like. Notwithstanding the definition above, one of either PG² or PG³ in compounds of formula A may be hydrogen. Also notwithstanding the definitions above, the —N(PG²)(PG³) moiety of formula A may be azido. According to one aspect of the invention, the —N(PG²)(PG³) moiety of formula A, is phthalimido. According to another aspect of the invention, at step S-13, a compound of formula Z is treated with potassium phthalimide to generate compounds of formula A in which the —N(PG²)(PG³) moiety is phthalimido.

In certain embodiments, step S-13 is performed with heating. In other embodiments, the reaction is conducted at a temperature that is between about 40° C. and about 110° C. In other embodiments, the reaction is run at about 85° C.

In certain embodiments, step S-13 is conducted in the presence of a polar aprotic solvent. Exemplary polar aprotic solvents include dimethylformamide (DMF), N-methylpyrrolidine (NMP), dimethylacetamide (DMA), dioxane, tetrahydrofuran (THF), and dimethylsulfoxide (DMSO). In certain embodiments, the reaction is conducted in dimethylformamide (DMF), N-methylpyrrolidine (NMP), or dimethylacetamide (DMA). In other embodiments, the reaction is conducted in DMF.

Compounds of formula A may be transformed to compounds of formulae II and II•HX according to steps S-8 and S-9 as described in detail above and herein with respect to Scheme I.

According to another aspect, the present invention provides a method for preparing a compound of formula II•HX: 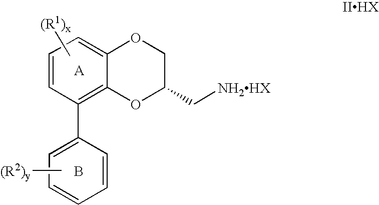
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, Cl aliphatic or C₁₋₆ fluoroaliphatic;
each R² is independently —R, -Ph, —CN, halogen, —OR, —C(O)NH₂, —C(O)OR, —NHC(O)R, —SO₂R, or —NHSO₂R; and
X is the anion of a suitable acid,
   comprising the steps of:
(a) providing a compound of formula II: 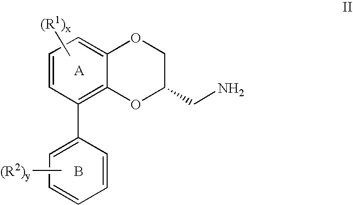
   wherein:
   x is 0-3;
   y is 0-5;
   each R¹ is independently —R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic; and
   each R² is independently R, -Ph, —CN, halogen, —OR, —C(O)NH₂, —C(O)OR, —NHC(O)R, —SO₂R, or —NHSO₂R,
      and
(b) reacting said compound of formula II with suitable acid of formula HX to form a compound of formula II•X.

As defined above, in compounds of formulae II and II•HX, x is 0-3, y is 0-5, each R¹ is independently —R, -Ph, —CN, halogen, —OR, —C(O)NH₂, —C(O)OR, —NHC(O)R, —SO₂R, or —NHSO₂R, each R is independently hydrogen, C₁₋₆ aliphatic or C₁₋₆ fluoroaliphatic, and each R² is independently R, -Ph, —CN, halogen, —OR, —C(O)NH₂, —C(O)OR, —NHC(O)R, —SO₂R, or —NHSO₂R. In certain embodiments, x is 1-2. In other embodiments, x is 1. In certain embodiments, y is 2-3. In other embodiments, y is 2. In certain embodiments, R¹ is halogen. In other embodiments, R¹ is fluoro. In certain embodiments, R² is halogen or C₁₋₆ aliphatic. In other embodiments, R² is chloro. In certain embodiments, ring A is substituted with an R¹ group at the open meta position relative to the carbon bearing ring B. In other embodiments, Ring B is substituted with at least one R² group at a position ortho to the carbon bearing ring A. In yet other embodiments, ring B is substituted at each position ortho to the carbon bearing ring A with an R² group.

In certain embodiments, the compound of formula II, is selected from those depicted in table 2, below.

TABLE 2

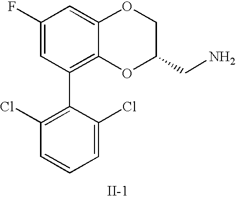

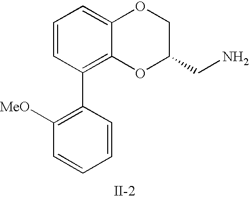

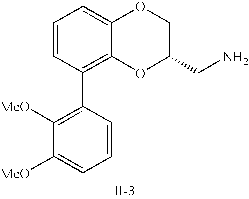

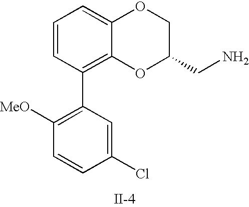

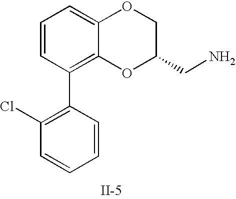

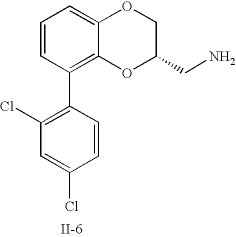

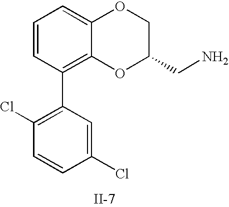

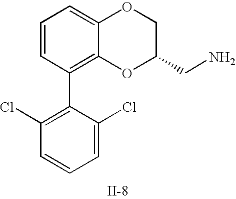

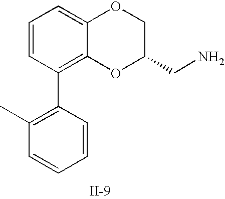

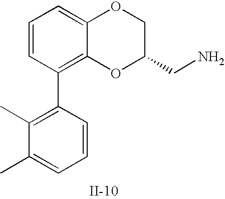

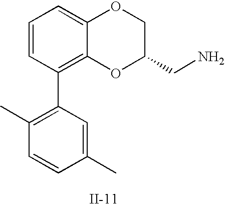

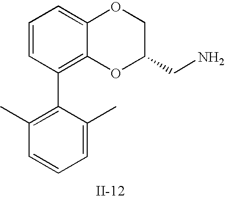

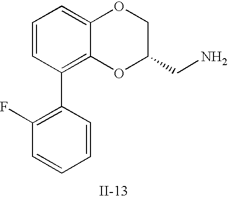

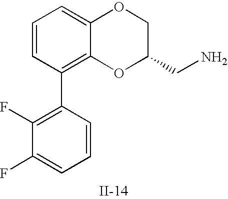

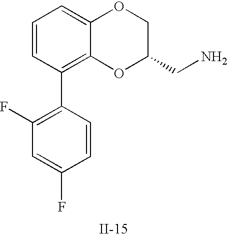

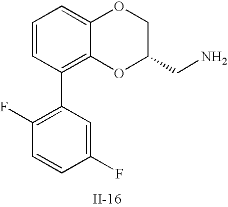

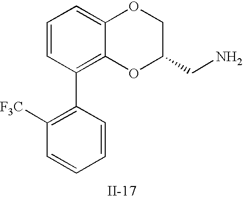

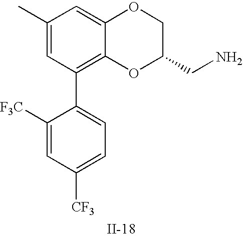

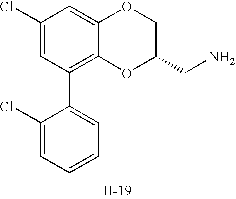

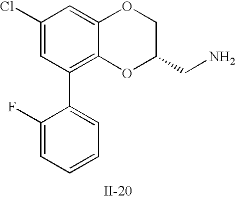

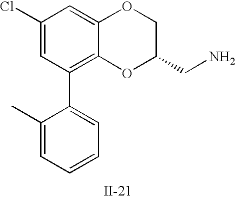

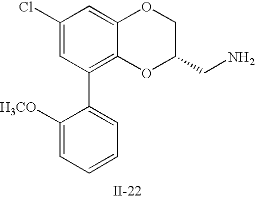

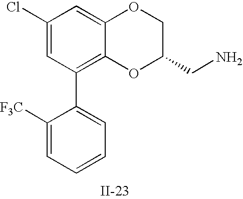

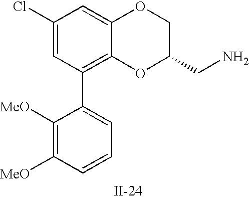

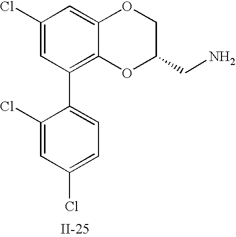

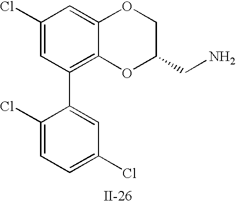

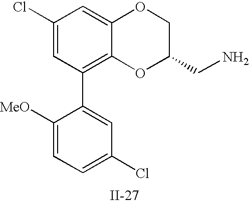

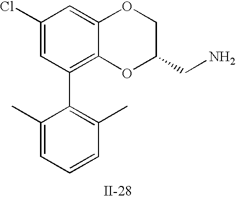

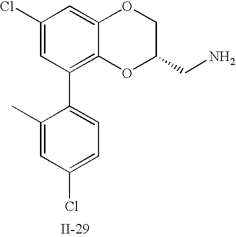

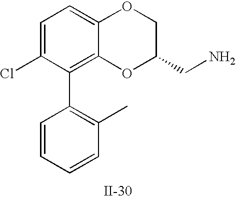

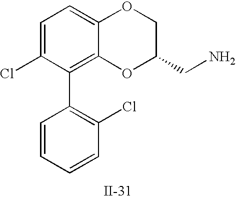

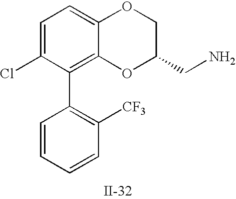

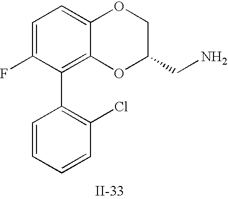

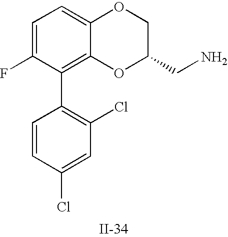

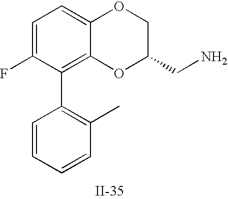

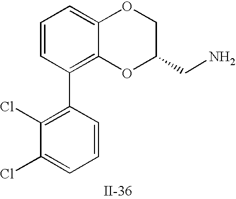

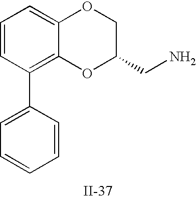

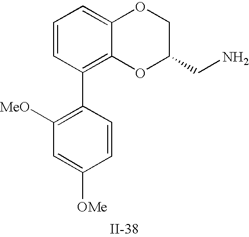

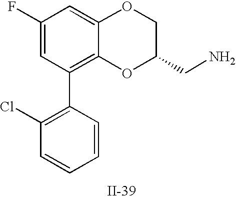

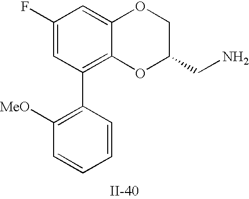

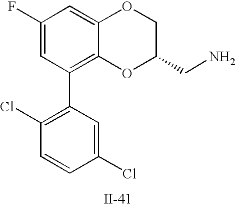

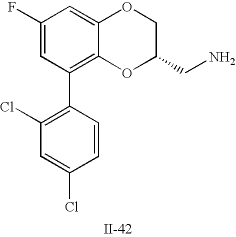

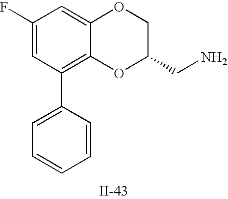

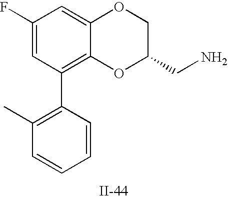

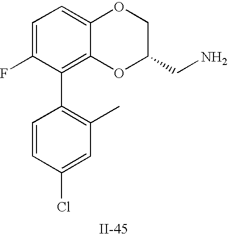

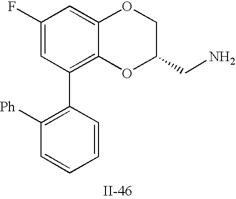

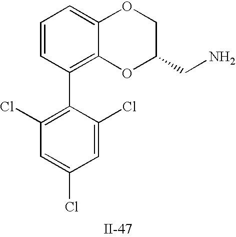

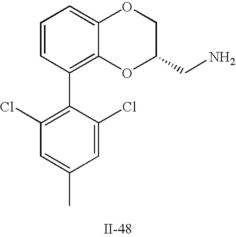

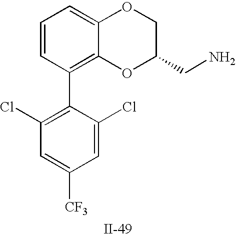

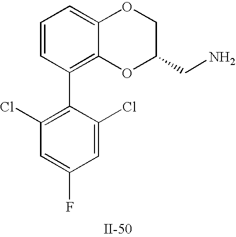

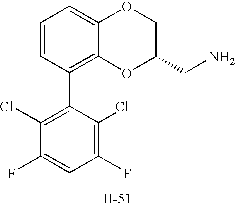

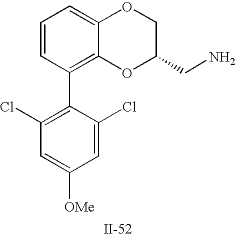

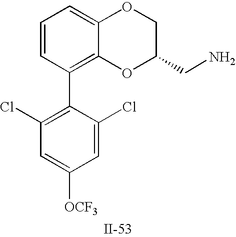

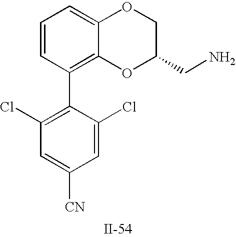

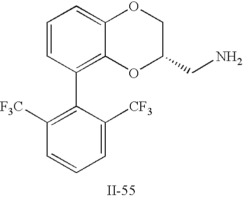

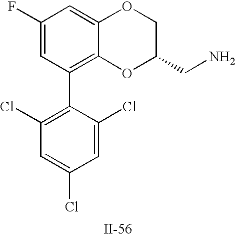

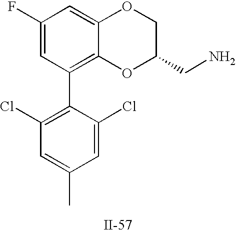

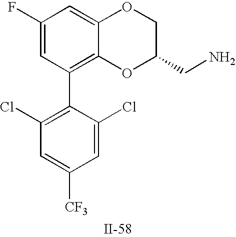

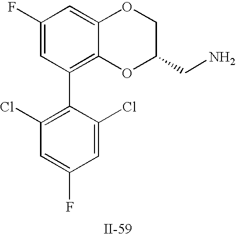

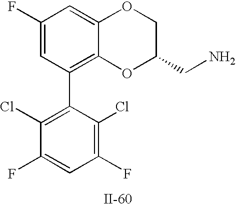

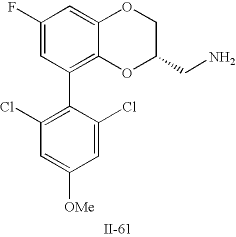

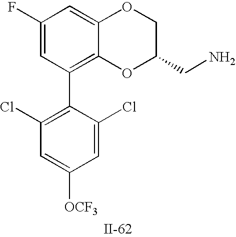

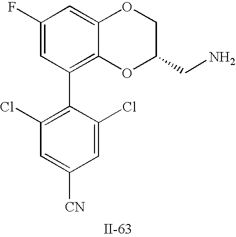

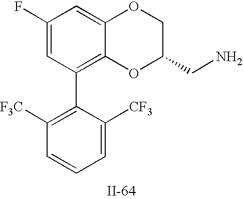

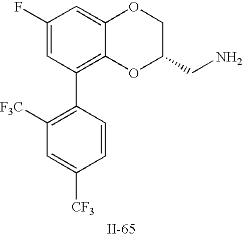

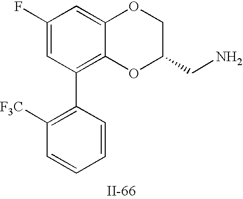

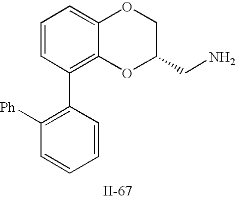

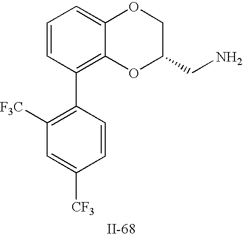

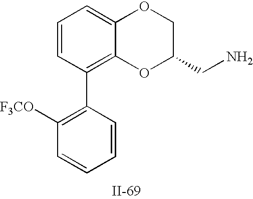

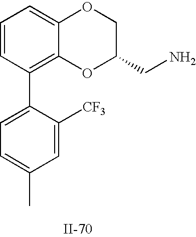

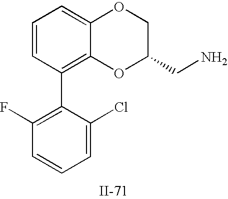

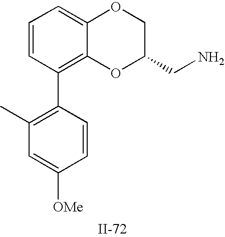

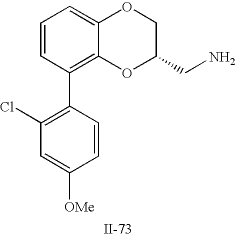

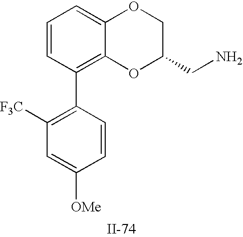

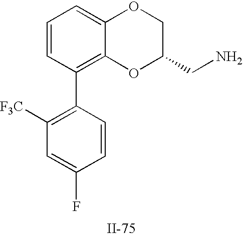

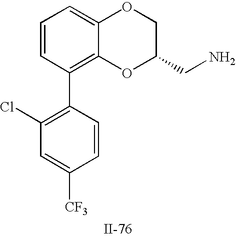

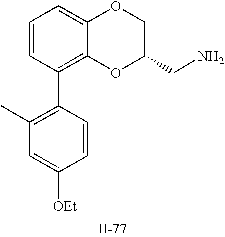

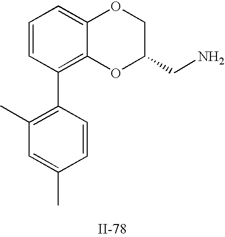

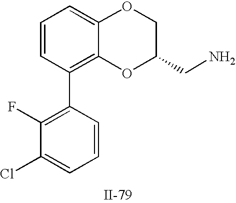

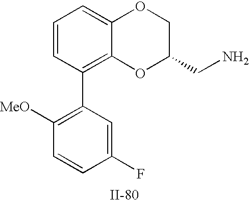

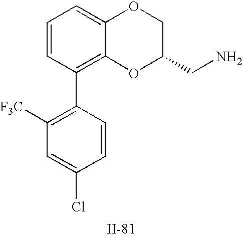

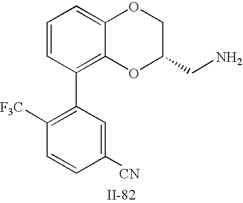

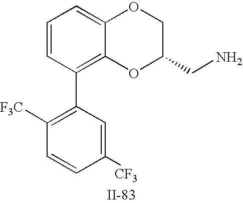

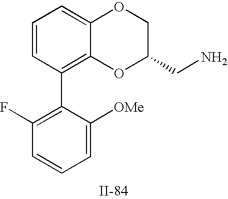

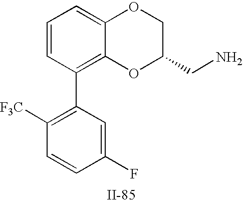

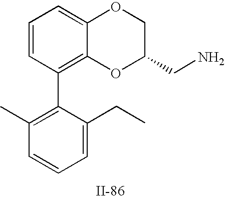

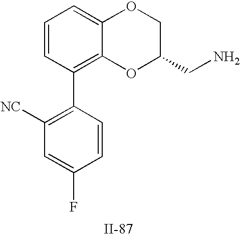

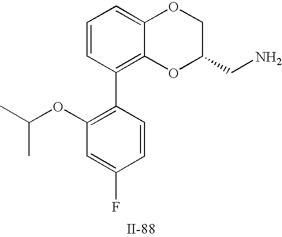

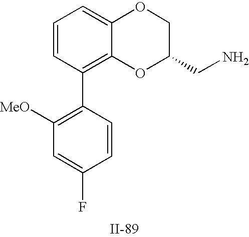

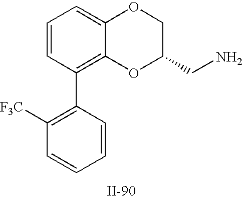

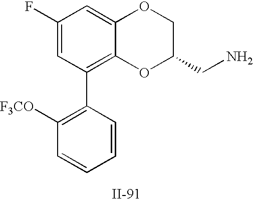

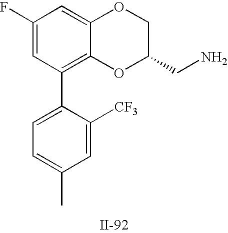

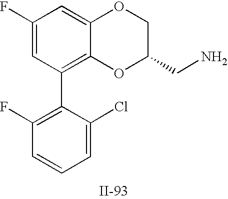

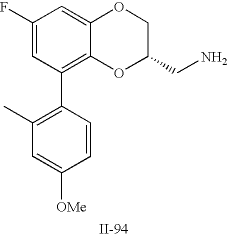

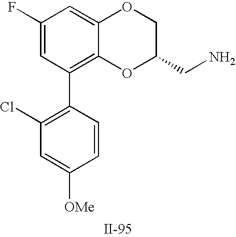

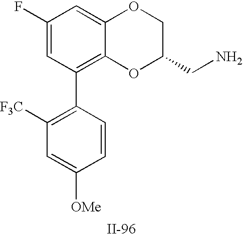

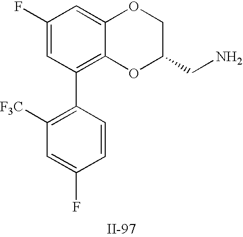

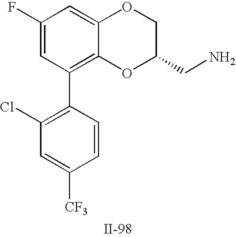

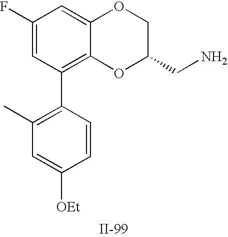

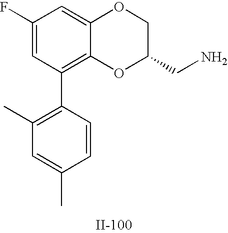

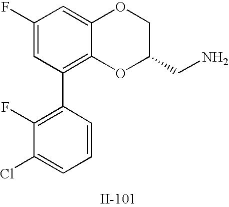

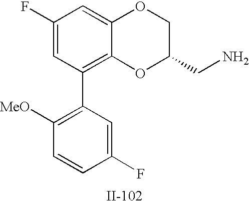

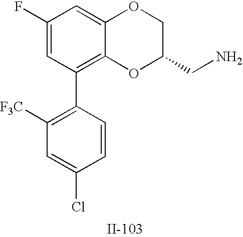

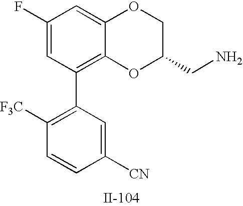

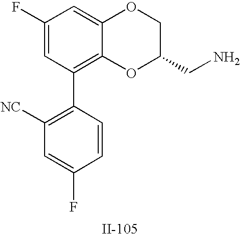

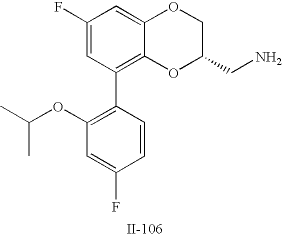

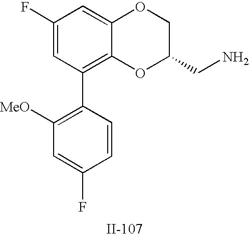

In other embodiments, the compound of formula II is selected from II-1, II-8, and II-28. In yet another embodiment, the compound of formula II is II-1.

As defined above, HX in the reaction step above and in compounds of formula II•HX is a suitable Brønsted acid. Exemplary acids include hydrogen halides, carboxylic acids, sulfonic acids, sulfuric acid, and phosphoric acid. According to one aspect of the present invention, a compound of formula II is treated with HCl to form a compound of formula II•HX wherein X is Cl. In certain embodiments, where the acid is HCl, the acid is introduced into the medium containing the compound of formula II in gaseous form. In other embodiments, the acid is introduced into the medium containing the compound of formula II as a solution in methanol, ethanol, isopropanol, or water. In yet other embodiments, the acid is introduced into the medium containing the compound of formula II as a solution in isopropanol. In certain embodiments, the medium containing the compound of formula II is isopropanol.

In certain embodiments, the compound of formula II•HX is selected from the group of compounds formed by combining those compounds of formula II depicted in Table 2 with a suitable Brønsted acid. In other embodiments, the compound of formula II•HX is selected from those salts formed by combining compound II-1 with a suitable Brønsted acid. In yet another embodiment, the compound of formula II•HX is the HCl salt of compound II-1.

In certain embodiments, the compound of formula II•HX is isolated by crystallization. In other embodiments, this crystallization step serves as the only isolation or purification step for compounds of this formula. In still other embodiments, the crystallization is optionally repeated until the compound of formula II•HX is of desired purity. In yet other embodiments, this crystallization increases the enantiomeric excess of the crystalline product, and is optionally conducted by seeding the solution of the enantiomers of formula II•HX with one or more crystals of the same that is enriched in the desired enantiomeric form.

According to another embodiment, the present invention provides a method for preparing a compound of formula II: 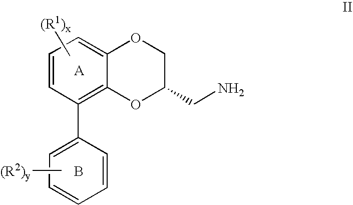
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic; and
each R² is independently R, -Ph, —CN, halogen, —OR, —C(O)NH₂, —C(O)OR, —NHC(O)R, —SO₂R, or —NHSO₂R,
   comprising the steps of:
(a) providing a compound of formula A: 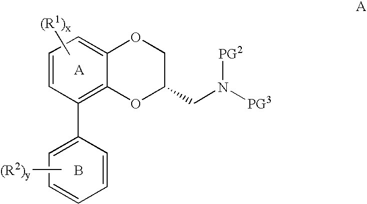
   wherein:
   x is 0-3;
   y is 0-5;
   each R¹ is independently —R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic;
   each R² is independently R, -Ph, —CN, halogen, —OR, —C(O)NH₂, —C(O)OR, —NHC(O)R, —SO₂R, or —NHSO₂R; and
   PG² and PG³ are each suitable amino protecting groups,
      and
(b) deprotecting the protected amine moiety of said compound of formula A to form a compound of formula II.

For compounds of formula A, each of x, y, R¹, and R² are as defined above in embodiments and subembodiments for compounds of formula II and II•HX. As defined above, the PG² and PG³ groups of compounds of formula A are each suitable amino protecting groups. Protected amines are well known in the art and include those described in detail in Greene (1999). Suitable mono-protected amines further include, but are not limited to, aralkylamines, carbamates, allyl amines, amides, and the like. Examples of suitable mono-protected amino moieties include t-butyloxycarbonylamino (—NHBOC), ethyloxycarbonylamino, methyloxycarbonylamino, trichloroethyloxycarbonylamino, allyloxycarbonylamino (-NHAlloc), benzyloxocarbonylamino (-NHCBZ), allylamino, benzylamino (-NHBn), fluorenylmethylcarbonyl (-NHFmoc), formamido, acetamido, chloroacetamido, dichloroacetamido, trichloroacetamido, phenylacetamido, trifluoroacetamido, benzamido, t-butyldiphenylsilyl, and the like. Suitable di-protected amines include amines that are substituted with two substituents independently selected from those described above as mono-protected amines, and further include cyclic imides, such as phthalimide, maleimide, succinimide, and the like. Suitable di-protected amines also include pyrroles and the like, and 2,2,5,5-tetramethyl-[1,2,5]azadisilolidine and the like. Notwithstanding the definition above, one of either PG² or PG³ in compounds of formula A may be hydrogen. Also notwithstanding the definitions above, the —N(PG²)(PG³) moiety of formula A may be azido. According to one aspect of the invention, the —N(PG²)(PG³) moiety of formula A is phthalimido. According to one aspect of the present invention, the compound of formula A is

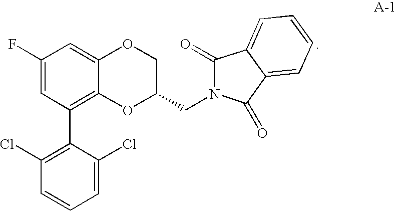

In this step, removal of the PG² and PG³ protecting groups in compounds of formula A affords the free amine-containing compounds of formula II. Procedures for the removal of suitable amino protecting groups are well known in the art; see Green (1999). In certain embodiments, where the —N(PG²)(PG³) moiety of formula A is phthalimido, PG² and PG³ are removed by treatment with hydrazine or methylamine. In other embodiments, where the —N(PG²)(PG³) moiety of formula A is phthalimido, PG² and PG³ are removed by treatment with hydrazine.

In certain embodiments, the deprotection is formed in the presence of a suitable medium. A suitable medium is a solvent or a solvent mixture that, in combination with the combined reacting partners and reagents, facilitates the progress of the reaction therebetween. The suitable solvent may solubilize one or more of the reaction components, or, alternatively, the suitable solvent may facilitate the suspension of one or more of the reaction components; see, generally, March (2001). In certain embodiments, this transformation is conducted with ethanol, methanol, isopropanol, or tetrahydrofuran as solvent, or with mixtures of the aforementioned solvents and/or water. In other embodiments, this transformation is conducted with ethanol as solvent. In certain embodiments, the reaction is conducted at a temperature between about 40° C. and about 90° C. In other embodiments the reaction is conducted with an ethanol-water mix as solvent at reflux.

According to one aspect of the present invention, a mixture of enantiomers of formula II is allowed to react with an enantiomerically enriched chiral acid, and the diastereomeric excess of the resulting salts is increased by selective crystallization of one of the diastereomers over the others. According to yet another aspect of the present invention, the chiral acid employed in the aforementioned crystallizations is dibenzoyltartaric acid. In certain embodiments, the diastereomeric salts are formed by combining enantiomers of formula II with enantioenriched chiral acid in tetrahydrofuran, isopropanol, ethanol, water, or mixtures thereof, followed by optional heating to temperatures as high as the reflex temperature of the solvent used. In other embodiments, the aforementioned diastereomeric salts are formed in refluxing tetrahydrofuran. In certain embodiments, the crystallization of diastereomeric salts of compounds of formula II is from a solution in tetrahydrofuran, isopropanol, ethanol, water, or mixtures thereof. In other embodiments, said crystallization is from a solution in tetrahydrofuran. In yet other embodiments, the crystallization occurs on cooling of a heated solution of the salt. In certain embodiments, the solution is heated as high as the reflux temperature of the solvent and cooled to as low as 10° C. Compounds of formula II can be obtained from the diastereomeric salts by treatment with a suitable base in a suitable solvent. One of ordinary skill in the art will appreciate that a wide variety of bases and solvents are appropriate for this purpose, thus a large variety thereof is envisioned. In certain embodiments, the suitable base is sodium hydroxide and the suitable solvent is selected from water, tert-butyl methyl ether, or a mixture thereof. According to another aspect of the present invention, the above-mentioned crystallization is optionally repeated to further increase the diastereomeric enrichment of the compounds that are being crystallized.

According to another embodiment, the present invention provides a method for preparing a compound of formula A: 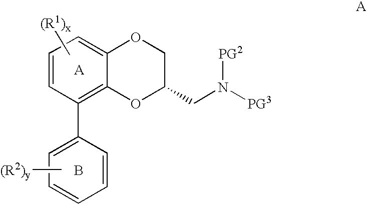
wherein:
x is 0-3;
y is 0-5;
each R¹ is independently —R, -Ph, —CN, halogen, —OR, C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R; and
PG² and PG³ are each suitable amino protecting groups,
   comprising the steps of:
(a) providing a compound of formula B: 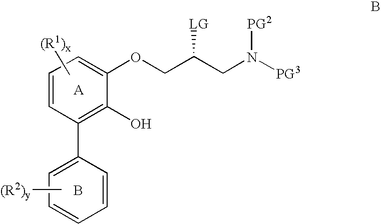
   wherein:
   x is 0-3;
   y is 0-5;
   each R¹ is independently —R, -Ph, —CN, halogen, —OR, —C(O)NH₂, —C(O)OR, —NHC(O)R, -SO₂R, or —NHSO₂R;
   each R is independently hydrogen, C₁₋₆ aliphatic or C₁₋₆ fluoroaliphatic;
   each R² is independently R, -Ph, —CN, halogen, —OR, —C(O)NH₂, —C(O)OR, —NHC(O)R, —SO₂R, or —NHSO₂R;
   PG² and PG³ are each suitable amino protecting groups; and
   LG is a suitable leaving group,
      and
(b) allowing said compound of formula B to cyclize to form a compound of formula A.

For compounds of formula B, each of x, y, R¹, and R² are as defined above in embodiments and subembodiments for compounds of formula II and II•HX. Similarly, for compounds of formula B, the PG² and PG³ groups are as defined above in embodiments and subembodiments for compounds of formula A.

As defined above, the LG group in compounds of formula B is a suitable leaving group. Suitable leaving groups are well known in the art, e.g., see, March (2001). Such leaving groups include, but are not limited to, halogen, alkoxy, sulphonyloxy, optionally substituted alkylsulphonyloxy, optionally substituted alkenylsulfonyloxy, optionally substituted arylsulfonyloxy, and diazonium moieties. Examples of suitable leaving groups include chloro, iodo, bromo, fluoro, methanesulfonyloxy (mesyloxy), p-toluenesulfonyloxy (tosyloxy), trifluoromethanesulfonyloxy (triflyloxy), nitro-phenylsulfonyloxy (nosyloxy), and bromo-phenylsulfonyloxy (brosyloxy). In certain embodiments, LG is halogen. In other embodiments, LG is an optionally substituted alkylsulphonyloxy, optionally substituted alkenylsulfonyloxy, or optionally substituted arylsulfonyloxy. In certain embodiments, the suitable leaving group is optionally substituted arylsulfonyloxy or alkylsulphonyloxy. In other embodiments, the leaving group is tosyloxy or mesyloxy. In one embodiment, the leaving group is mesyloxy. According to one aspect of the present invention, the compound of formula B is

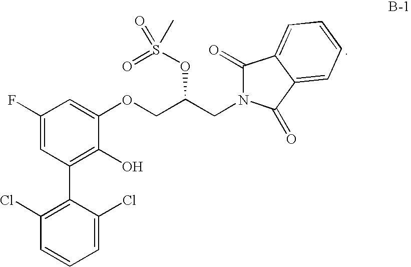

At this step, a compound of formula B is allowed to cyclize to afford a compound of formula A. One of ordinary skill in the art would recognize that a wide variety of reaction conditions are useful for promoting this reaction, therefore a wide variety of reaction conditions are contemplated. For example, the reaction may be conducted with or without thermal excitation, with or without base catalysis, and in protic or aprotic media.

In certain embodiments, the cyclization reaction is performed in the presence of a suitable base. A suitable base is a Brønsted or Lewis basic species that, in combination with the combined reacting partners and reagents, facilitates the progress of the reaction therebetween; see generally, March (2001). According to one aspect of the invention, the reaction is promoted by the addition of potassium carbonate, lithium diisopropylamide, or lithium hexamethyldisilazide to a compound of formula B. According to another aspect of the invention, the reaction is promoted by the addition of potassium carbonate.

In certain embodiments, the cyclization reaction is performed in a suitable medium. In certain embodiments, the reaction is conducted with dimethylformamide, N-methylpyrrolidone, or dimethylamine as solvent. In other embodiments, the reaction is conducted with dimethylformamide as solvent.

In certain embodiments, the cyclization reaction is conducted at a temperature between about 10° C. and about 60° C. In other embodiments, the cyclization reaction is conducted at a temperature of between about 20° C. and about 25° C.

According to one aspect of the present invention, a mixture of enantiomers of formula A is dissolved in a suitable solvent and crystallized therefrom to afford a crystalline product that is further enriched in a single enantiomer. In certain embodiments, the suitable solvent is selected from toluene, ethyl acetate, dimethylformamide, and tetrahydrofuran. In other embodiments, the suitable solvent is toluene. In other embodiments, the mixture of enantiomers of formula A is dissolved in a suitable solvent at a temperature between about 70° C. and about 90° C. In certain embodiments, the crystallization occurs on cooling of a heated solution of enantiomers of formula A. In still other embodiments, the crystallization is performed by seeding the solution of enantiomers with a crystal that is enantioenriched in the enantiomer of interest. According to another aspect of the present invention, the above-mentioned selective crystallization is optionally repeated to further increase the enantiomeric enrichment of the compounds that are being crystallized. It will be appreciated that enantioenrichment of the crystals also results in enrichment of the opposite enantiomer of the mother liquor. Accordingly, it is contemplated that both enantiomers may be obtained in enriched form.

Yet another aspect of the present invention provides a method for preparing a compound of formula B: 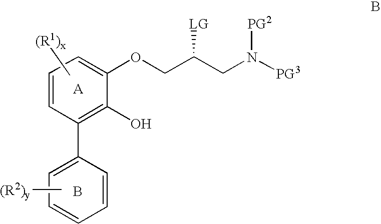
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R;
PG² and PG³ are each suitable amino protecting groups; and
LG is a suitable leaving group,
   comprising the steps of:
(a) providing a compound of formula C: 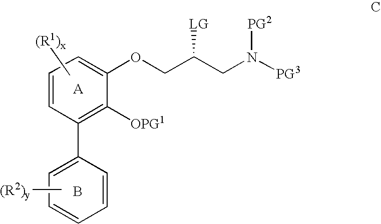
   wherein:
   x is 0-3;
   y is 0-5;
   each R¹ is independently —R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic;
   each R² is independently R, -Ph, —CN, halogen, —OR, —C(O)NH₂, —C(O)OR, —NHC(O)R, —SO₂R, or —NHSO₂R;
   PG¹ is a suitable hydroxyl protecting group;
   PG² and PG³ are each suitable amino protecting groups; and
   LG is a suitable leaving group,
      and
(b) deprotecting the protected hydroxyl moiety of said compound of formula C to form a compound of formula B.

For compounds of formula C, each of x, y, R¹, and R² are as defined above in embodiments and subembodiments for compounds of formula II and II•HX. Similarly, for compounds of formula C, the PG² and PG³ groups are as defined above in embodiments and subembodiments for compounds of formula A, and the LG group is as defined above in embodiments and subembodiments for compounds of formula B. As defined above, in compounds of formula C, the PG¹ group is a suitable hydroxyl protecting group. Protected hydroxyl groups (corresponding to OPG¹ of formula C) are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitably protected hydroxyl groups further include, but are not limited to, esters, carbonates, sulfonates allyl ethers, ethers, silyl ethers, alkyl ethers, arylalkyl ethers, and alkoxyalkyl ethers. Examples of suitable esters include formates, acetates, proprionates, pentanoates, crotonates, and benzoates. Specific examples of suitable esters include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benylbenzoate, 2,4,6-trimethylbenzoate. Examples of suitable carbonates include 9-fluorenylmethyl, ethyl, 2,2,2-.trichloroethyl, 2-(trimethylsilyl)ethyl, 2-(phenylsulfonyl)ethyl, vinyl, allyl, and p-nitrobenzyl carbonate. Examples of suitable silyl ethers include trimethylsilyl, triethylsilyl, t-butyldimethylsilyl, t-butyldiphenylsilyl, triisopropylsilyl ether, and other trialkylsilyl ethers. Examples of suitable alkyl ethers include methyl, benzyl, p-methoxybenzyl, 3,4-dimethoxybenzyl, trityl, t-butyl, and allyl ether, or derivatives thereof. Alkoxyalkyl ethers include acetals such as methoxymethyl, methylthiomethyl, (2-methoxyethoxy)methyl, benzyloxymethyl, beta-trimethylsilyl)ethoxymethyl, and tetrahydropyran-2-yl ether. Examples of suitable arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers. According to one aspect of the present invention, the PG¹ group of formula C is methyl. In one embodiment, the leaving group is mesyloxy. According to one aspect of the present invention, the compound of formula C is

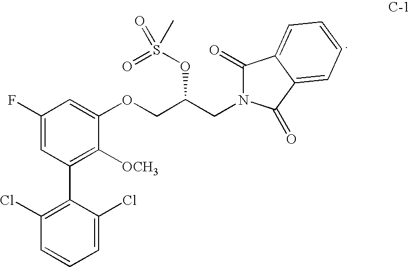

At this step, removal of the PG¹ protecting group in compounds of formula C affords the free phenol-containing compounds of formula B. Procedures for the removal of suitable hydroxyl protecting groups are well known in the art; see Green (1999). In certain embodiments, where PG¹ is methyl, PG¹ is removed by treatment of a compound of formula C with BBr₃, iodotrimethylsilane, or a combination of BCl₃ and LiI. According to one aspect of the present invention, where PG¹ is methyl, PG¹ is removed by treatment of a compound of formula C with BBr₃.

In certain embodiments, the deprotection is performed in a suitable medium. In certain embodiments this step is conducted using toluene, dichloromethane, or isopropyl acetate as solvent. In other embodiments, this step is conducted using toluene as solvent.

In certain embodiments, the reaction is conducted at a temperature between about −20° C. and about 40° C. In other embodiments, the cyclization reaction is conducted at a temperature of between about 20° C. and about 25° C.

According to another embodiment, the present invention provides a method for preparing a compound of formula C: 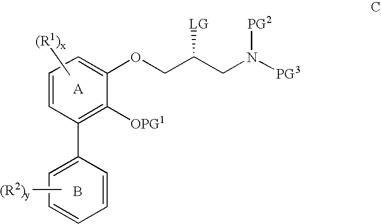
wherein:
x is 0-3;
y is 0-5;
each R¹ is independently —R, -Ph, —CN, halogen, —OR, —C(O)NH₂, C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R;
PG¹ is a suitable hydroxyl protecting group;
PG² and PG³ are each suitable amino protecting groups; and
LG is a suitable leaving group,
   comprising the steps of:
(a) providing a compound of formula D: 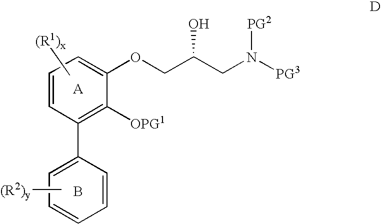
   wherein:
   x is 0-3;
   y is 0-5;
   each R¹ is independently —R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic;
   each R² is independently R, -Ph, —CN, halogen, —OR, —C(O)NH₂, —C(O)OR, —NHC(O)R, —SO₂R, or —NHSO₂R;
   PG¹ is a suitable hydroxyl protecting group; and
   PG² and PG³ are each suitable amino protecting groups,
      and
(b) converting the free hydroxyl moiety of said compound of formula D into a suitable leaving group to afford a compound of formula C.

For compounds of formula D, each of x, y, R¹, and R² are as defined above in embodiments and subembodiments for compounds of formula II and II•HX. Similarly, for compounds of formula D, the PG² and PG³ groups are as defined above in embodiments and subembodiments for compounds of formula A, and the PG¹ group is as defined above in embodiments and subembodiments for compounds of formula C. According to one aspect of the present invention, the compound of formula D is

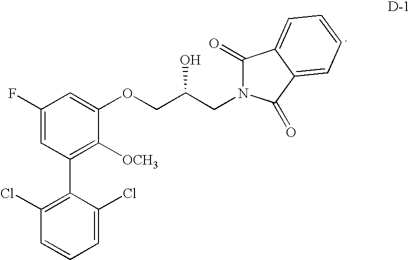

In this step, the hydroxyl group of compounds of formula D is activated such that it becomes leaving group LG that is subject to nucleophilic displacement. A suitable “leaving group” that is “subject to nucleophilic displacement” is a chemical group that is readily displaced by a desired incoming nucleophilic chemical entity. According to one aspect of the present invention, a compound of formula D is allowed to react with methanesulfonyl chloride (mesyl chloride) to afford a compound of formula C in which LG is methanesulfonyloxy (mesyloxy).

In certain embodiments this reaction is performed in a suitable medium. In certain embodiments, the suitable medium is in tetrahydrofuran (THF), dichloromethane, acetonitrile, or isopropyl acetate. In other embodiments the suitable medium is THF.

According to one aspect of the present invention, the reaction is run in the presence of triethylamine (TEA).

In certain embodiments, the reaction is run at a temperature that is between about −20° C. and about 40° C. In other embodiments, the reaction is conducted at a temperature of about 0° C.

In certain embodiments, the present invention provides a method for preparing a compound of formula D: 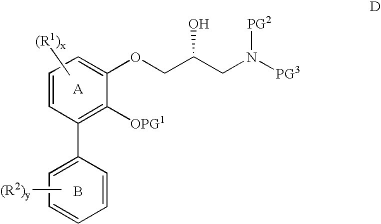
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R;
PG¹ is a suitable hydroxyl protecting group; and
PG² and PG³ are each suitable amino protecting groups,
   comprising the steps of:
(a) providing a compound of formula E: 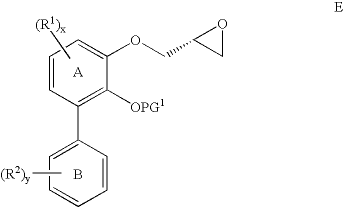
   wherein:
   x is 0-3;
   y is 0-5;
   each R¹ is independently —R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic;
   each R² is independently R, -Ph, —CN, halogen, —OR, —C(O)NH₂, —C(O)OR, —NHC(O)R, —SO₂R, or —NHSO₂R; and
   PG¹ is a suitable hydroxyl protecting group,
      and
(b) treating the compound of formula E with a suitable amine to afford a compound of formula D.

For compounds of formula E, each of x, y, R¹, and R² are as defined above in embodiments and subembodiments for compounds of formula II and II•HX. Similarly, for compounds of formula E, the PG¹ group is as defined above in embodiments and subembodiments for compounds of formula C. According to one aspect of the present invention, the compound of formula E is

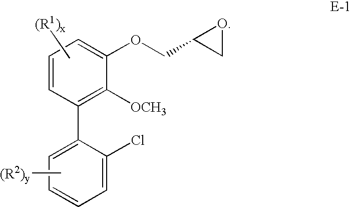

In this step, a protected amine moiety is introduced via epoxide-opening to afford compounds of formula D. According to one aspect of the invention, in this step, a compound of formula E is treated with phthalimide in the presence of a suitable base to generate a compound of formula D. In certain embodiments, a compound of formula E is treated with potassium phthalimide to generate compounds of formula D in which the —N(PG²)(PG³) moiety is phthalimido.

In other embodiments, this step is performed with heating. In certain embodiments, the reaction is conducted at a temperature that is between about 40° C. and about 110° C. In other embodiments, the reaction is conducted at about 80° C.

In certain embodiments, the reaction is conducted in a suitable medium. In some embodiments, the suitable medium is dimethylformamide (DMF), N-methylpyrrolidine (NMP), or dimethylacetamide (DMA). In other embodiments, the reaction is conducted in DMF.

According to another embodiment, the present invention provides a method for preparing a compound of formula E: 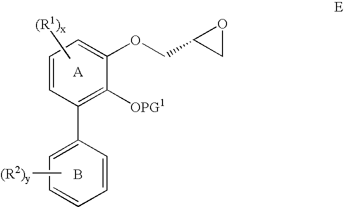
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R; and
PG¹ is a suitable hydroxyl protecting group,
   comprising the steps of:
(a) providing a compound of formula F: 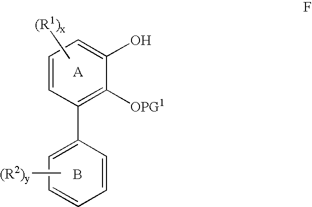
   wherein:
   x is 0-3;
   y is 0-5;
   each R¹ is independently —R, -Ph, —CN, halogen, —OR, —C(O)NH₂, C(O)OR, —NHC(O)R, —SO₂R, or —NHSO₂R;
   each R is independently hydrogen, C₁₋₆ aliphatic or C₁₋₆ fluoroaliphatic;
   each R² is independently R, -Ph, —CN, halogen, —OR, —C(O)NH₂, —C(O)OR, —NHC(O)R, —SO₂R, or —NHSO₂R; and
   PG¹ is a suitable hydroxyl protecting group,
      and
(b) glycidating said compound of formula F to form a compound of formula E.

For compounds of formula F, each of x, y, R¹, and R² are as defined above in embodiments and subembodiments for compounds of formula II and II•HX. Similarly, for compounds of formula F, the PG¹ group is as defined above in embodiments and subembodiments for compounds of formula C. According to one aspect of the present invention, the compound of formula F is

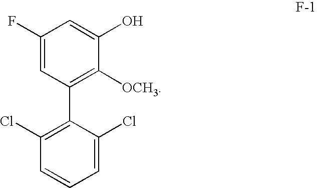

In this step, a compound of formula F is glycidated on the phenol oxygen of compounds of formula F. Exemplary reagents that may be used to promote glycidation include epichlorohydrin, epibromohydrin, oxiranylmethyl p-toluenesulfonate (also called: oxiranylmethyl tosylate or glycidyl tosylate), oxiranylmethyl methanesulfonate (oxiranylmethyl mesylate or glycidyl mesylate), and oxiranylmethyl trifluoromethanesulfonate (oxiranylmethyl triflate or glycidyl triflate). According to one aspect of the present invention, the activated glycidol equivalent (also referred to herein as the suitable glycidating reagent) is gycidyl tosylate.

In certain embodiments, in this step, a compound of formula F is treated with a suitable base and a suitable glycidating reagent to afford a compound of formula E. In certain embodiments, the base employed is selected from sodium hydroxide (NaOH), potassium carbonate (K₂CO₃), potassium tert-butoxide (KOtBu), lithium diisopropylamide (LDA), lithium hexamethyldisilazide (LHMDS), or sodium hydride (NaOH). According to one aspect of the present invention, the base is potassium tert-butoxide.

According to one aspect of the present invention, the glycidation is conducted in a suitable medium. In certain embodiments, the reaction is conducted in dimethylformamide (DMF), N-methylpyrrolidine (NMP), or dimethylacetamide (DMA). In other embodiments, DMF is employed as a solvent.

In certain embodiments, the glycidation reaction is heated. In certain embodiments, the reaction is conducted at a temperature that is between about 20° C. and about 100° C. In other embodiments, the glycidation reaction is conducted at a temperature that is between about 40° C. and about 50° C.

One of ordinary skill in the art will recognize that the activated glycidol equivalents contain a stereogenic carbon and accordingly, compounds of formula E contain a steregenic carbon corresponding thereto. In certain embodiments, the glycidol equivalent employed in this step is enantiomerically enriched, and accordingly, the mixture of enantiomers of formula E that are generated in this step is enantiomerically enriched. While a single stereochemical isomer is depicted in formula E, it will be appreciated that mixtures of enantiomers of compounds of this formula are accessible enriched in either enantiomer via the present invention.

In certain embodiments, the glycidaton step and the subsequent epoxide-opening step are conducted without isolating compounds of formula E. Accordingly, one aspect of the present invention is a procedure of glycidation followed by epoxide-opening to introduce a protected amine moiety without isolation of the intermediate glycidated species. In certain embodiments, the phthalimide is directly added to the reaction mixture in which the glycidated species was formed.

According to another embodiment, the present invention provides a method of obtaining a compound of formula F: 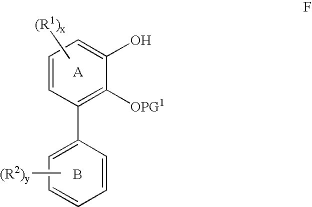
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —(O)NH₂, —C(O)OR, —NHC(O)R, —SO₂R, or —NHSO₂R; and
PG¹ is a suitable hydroxyl protecting group,
   comprising the steps of:
(a) providing a compound of formula G: 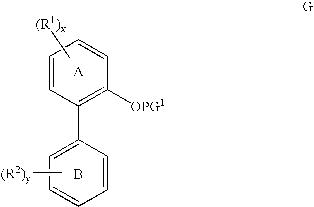
   wherein:
   x is 0-3;
   y is 0-5;
   each R¹ is independently —R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic;
   each R² is independently R, -Ph, —CN, halogen, —OR, —C(O)NH₂, —C(O)OR, —NHC(O)R, —SO₂R, or —NHSO₂R; and
   PG¹ is a suitable hydroxyl protecting group,
      and
(b) introducing a hydroxyl group ortho to the OPG¹ moiety in said compound of formula G to form a compound of formula F.

For compounds of formula G, each of x, y, R¹, and R² are as defined above in embodiments and subembodiments for compounds of formula II and II•HX. Similarly, for compounds of formula G, the PG¹ group is as defined above in embodiments and subembodiments for compounds of formula C. According to one aspect of the present invention, the compound of formula G is

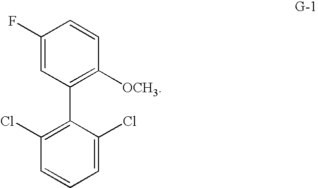

In this step, a hydroxyl group is introduced at the open ortho position relative to the OPG¹ group of formula G. One of ordinary skill in the art will recognize that there are a wide variety of reactions and reaction sequences that can be employed to accomplish this transformation; see generally, March ( 2001) and Larock (1999). Exemplary sequences include initial directed orthometallation followed by either (a) direct treatment with an electrophilic oxygen source; (b) treatment with a borate ester followed by oxidative workup of the resulting boronic ester or acid; or (c) treatment with a reagent that will allow the introduction of a formyl group (e.g., methyl formate, dimethylformamide) followed by subsequent Baeyer-Villiger reaction; for the above methods, see, e.g., Snieckus, (1990) and Schlosser (2005). Alternatively, direct orthoformylation may be utilized, followed by a Baeyer-Villiger reaction; see, e.g., Laird (1979) and Hofsløkken (1999). Another exemplary sequence involves halogenation followed by a metallation/transmetallation sequence to afford a boronic acid, boronic ester, or borane, followed by peroxide oxidation; see, generally, de Meijere (2004) and Snieckus (1990).

According to one aspect of the present invention, a compound of formula G is first brominated, then is metallated to afford an intermediate arylmetal compound that is allowed to react with a borate ester to afford, following aqueous workup, a boronic acid, which is subsequently oxidized to provide a phenol of formula F, as depicted in Scheme II, above. According to another aspect of the invention, the brominating agent is N-bromosuccinimide. In certain embodiments, the bromination is conducted in the presence of para-toluenesulfonic acid and acetic acid. According to yet another aspect of the invention, the metallation/transmetallation sequence involves initial magnesium-halogen exchange, followed by treatment with a trialkyl borate. In certain embodiments, the magnesium-halogen exchange is accomplished by treating the intermediate aryl bromide with isopropylmagnesium bromide. According to one aspect of the invention, the magnesium-halogen exchange is conducted in tetrahydrofuran (THF). In other embodiments, the trialkyl borate is triisopropylborate [B(OiPr)₃]. In certain embodiments, the metallation/transmetallation step is conducted at a temperature that is between about −20° C. and about 20° C. In other embodiments, the metallation/transmetallation step is conducted at a temperature that is between about 0° C. and about 5° C. In certain embodiments, the boronic acid is oxidized with hydrogen peroxide (H₂O₂) to afford compounds of formula F. In other embodiments, the boronic acid is oxidized with peroxyacetic acid (also called peracetic acid) or meta-chloroperoxybenzoic acid (mCPBA). One of ordinary skill in the art will recognize that standard procedures for magnesium-halogen exchange followed by transmetallation to a boron-containing entity, followed by oxidation to the phenol can be performed without isolation of the respective intermediate species.

According to another embodiment, the present invention provides a method of obtaining a compound of formula G: 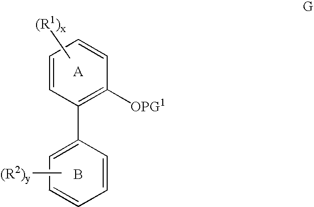
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R; and
PG¹ is a suitable hydroxyl protecting group,
   comprising the steps of:
(a) providing a compound of formula J: 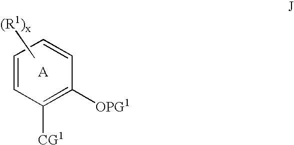
   wherein:
   x is 0-3;
   each R¹ is independently —R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic;
   PG¹ is a suitable hydroxyl protecting group; and
   CG¹ is a coupling group that facilitates transition metal-mediated Cₛₚ₂-Cₛₚ₂ coupling between the attached Cₛₚ₂ carbon and a Cₛₚ₂ carbon bearing a CG² coupling group,
      and
(b) coupling said compound of formula J with a compound of formula H: 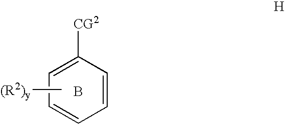
   wherein:
   y is 0-5;
   each R² is independently R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic; and
   CG² is a coupling group that facilitates transition metal-mediated Cₛₚ₂-Cₛₚ₂ coupling between the attached Cₛₚ₂ carbon and a Cₛₚ₂ carbon bearing a CG¹ coupling group;
      in the presence of a suitable transition metal to form a compound of formula G.

For compounds of formula J, each of x and R¹ are as defined above in embodiments and subembodiments for compounds of formula II and II•HX. Similarly, for compounds of formula J, the PG¹ group is as defined above in embodiments and subembodiments for compounds of formula C. For compounds of formula H, each of y and R² are as defined above in embodiments and subembodiments for compounds of formula II and II•HX. As defined above, the CG¹ group of compounds of formula J is a coupling group that facilitates transition metal-mediated Cₛₚ₂-Cₛₚ₂ coupling between the attached Cₛₚ₂ carbon and a Cₛₚ₂ carbon bearing a CG² coupling group. Similarly, as defined above, for compounds of formula H, CG² is a coupling group that facilitates transition metal-mediated Cₛₚ₂-Cₛₚ₂ coupling between the attached Cₛₚ₂ carbon and a Cₛₚ₂ carbon bearing a CG¹ coupling group.

In this coupling step, a compound of formula J is coupled to a compound of formula H, via a Cₛₚ₂-Cₛₚ₂ coupling reaction between the carbon centers bearing complementary coupling groups CG¹ and CG² to provide a compound of formula G. Suitable coupling reactions are well known to one of ordinary skill in the art and typically involve one of CG¹ or CG² being an electron-withdrawing group (e.g., Cl, Br, I, OTf, etc.), such that the resulting polar carbon-CG bond is susceptible to oxidative addition by an electron-rich metal (e.g., a low-valent palladium or nickel species), and the complementary coupling group being an electropositive group (e.g., boronic acids, boronic esters, boranes, stannanes, silyl species, zinc species, aluminum species, magnesium species, zirconium species, etc.), such that the carbon which bears the electropositive coupling group is susceptible to transfer to other electropositive species (e.g., a Pd^{II-IV} species or a Ni^{II-IV} species). Exemplary reactions include those described in Metal-Catalyzed Cross-Coupling Reactions, A. de Meijere and F. Diederich, Eds., 2^{nd} Edition, John Wiley & Sons, 2004. In certain embodiments, CG¹ in compounds of formula J is a boronic acid, a boronic ester, or a borane. In other embodiments, CG¹ in compounds of formula J is a boronic ester. According to one aspect of the present invention, CG¹ in compounds of formula J is a boronic acid. In certain embodiments, CG² in compounds of formula H is Br, I, or OTf. According to one aspect of the present invention, CG² in compounds of formula H is Br. According to one aspect of the present invention, the compound of formula J is 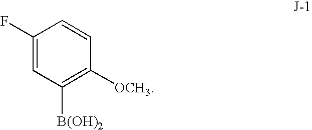
According to another aspect of the present invention, the compound of formula H is

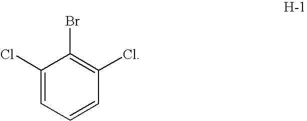

In certain embodiments, the coupling reaction is catalyzed by a palladium species. According to one aspect of the invention, the transformation is catalyzed by palladium tetrakis triphenylphosphine.

In certain embodiments, the coupling reaction is conducted in a suitable medium. In other embodiments, the coupling reaction is run with dimethoxyethane as solvent.

In certain embodiments, the reaction is heated. According to one aspect of the invention, the reaction is heated at reflux.

According to another aspect of the present invention, the reaction is run in the presence of sodium hydroxide.

According to another embodiment, the present invention provides a method for preparing a compound of formula D: 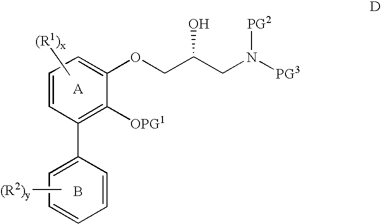
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R;
PG¹ is a suitable hydroxyl protecting group; and
PG² and PG³ are each suitable amino protecting groups,
   comprising the steps of:
(a) providing a compound of formula H: 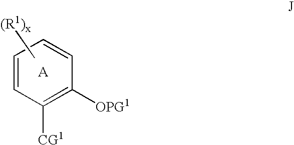
   wherein:
   x is 0-3;
   each R¹ is independently —R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic;
   PG¹ is a suitable hydroxyl protecting group; and
   CG¹ is a coupling group that facilitates transition metal-mediated Cₛₚ₂-Cₛₚ₂ coupling between the attached Cₛₚ₂ carbon and a Cₛₚ₂ carbon bearing a CG² coupling group,
(b) coupling said compound of formula J with a compound of formula H: 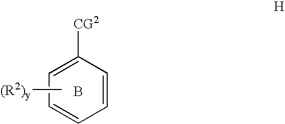
   wherein:
   y is 0-5;
   each R² is independently R, -Ph, —CN, halogen, —OR, —C(O)NH_{2,}—C(O)OR, —NHC(O)R, —SO₂R, or —NHSO₂R;
   each R is independently hydrogen, C₁₋₆ aliphatic or C₁₋₆ fluoroaliphatic; and

CG² is a coupling group that facilitates transition metal-mediated Cₛₚ₂-Cₛₚ₂ coupling between the attached Cₛₚ₂ carbon and a Cₛₚ₂ carbon bearing a CG¹ coupling group,
by the action of a suitable transition metal to provide a compound of formula G: 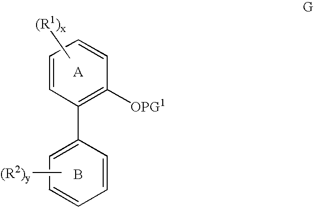
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R; and
PG¹ is a suitable hydroxyl protecting group,
(c) introducing a hydroxyl group ortho to the OPG¹ moiety in said compound of formula G to form a compound of formula F: 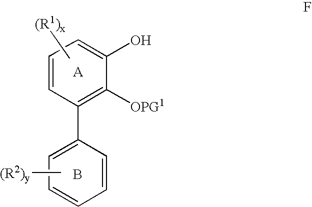
   wherein:
   x is 0-3;
   y is 0-5;
   each R¹ is independently —R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic;
   each R² is independently R, -Ph, —CN, halogen, —OR, —C(O)NH₂, —C(O)OR, —NHC(O)R, —SO₂R, or —NHSO₂R; and
   PG¹ is a suitable hydroxyl protecting group,
(d) glycidating said compound of formula F to form a compound of formula E: 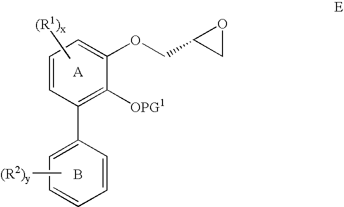
   wherein:
   x is 0-3;
   y is 0-5;
   each R¹ is independently —R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic;
   each R² is independently R, -Ph, —CN, halogen, —OR, —C(O)NH₂, —C(O)OR, —NHC(O)R, —SO₂R, or —NHSO₂R; and
   PG¹ is a suitable hydroxyl protecting group,
(e) treating the compound of formula E with a suitable amine to afford a compound of formula D.

In certain embodiments, the present invention provides a method for preparing a compound of formula II•HX: 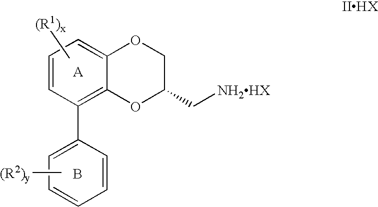
wherein:
x is 0-3;
y is 0-5;
each R¹ is independently —R, -Ph, —CN, halogen, —OR, C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R; and
X is the anion of a suitable acid,
   comprising the steps of:
(a) providing a compound of formula D: 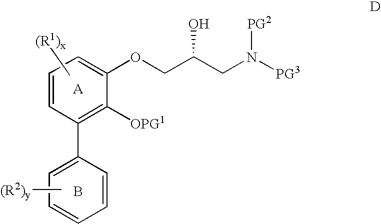
   wherein:
   x is 0-3;
   y is 0-5;
   each R¹ is independently —R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic;
   each R² is independently R, -Ph, CN, halogen, —OR, —C(O)NH₂, —C(O)OR, —NHC(O)R, —SO₂R, or —NHSO₂R;
   PG¹ is a suitable hydroxyl protecting group; and
   PG² and PG³ are each suitable amino protecting groups,
(b) converting the free hydroxyl moiety of said compound of formula D into a suitable leaving group to afford a compound of formula C: 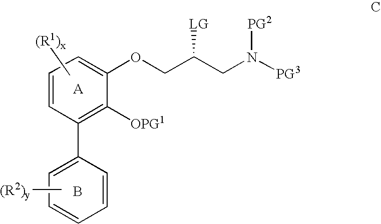
   wherein:
   x is 0-3;
   y is 0-5;
   each R¹ is independently —R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic;
   each R² is independently R, -Ph, —CN, halogen, —OR, —C(O)NH₂, —C(O)OR, —NHC(O)R, —SO₂R, or —NHSO₂R;
   PG¹ is a suitable hydroxyl protecting group;
   PG² and PG³ are each suitable amino protecting groups; and

LG is a suitable leaving group,
(c) deprotecting the protected hydroxyl moiety of said compound of formula C to form a compound of formula B: 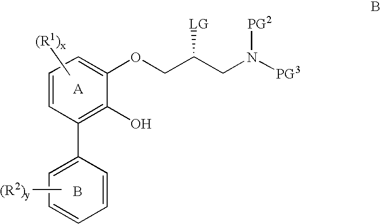
   wherein:
   x is 0-3;
   y is 0-5;
   each R¹ is independently —R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic;
   each R² is independently R, -Ph, —CN, halogen, —OR, —C(O)NH₂, —C(O)OR, —NHC(O)R, —SO₂R, or —NHSO₂R;
   PG² and PG³ are each suitable amino protecting groups; and
   LG is a suitable leaving group,
(d) allowing said compound of formula B to cyclize to form a compound of formula A: 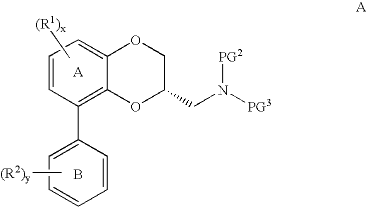
   wherein:
   x is 0-3;
   y is 0-5;
   each R¹ is independently —R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic;
   each R² is independently R, -Ph, —CN, halogen, —OR, —C(O)NH₂, —C(O)OR, —NHC(O)R, —SO₂R, or —NHSO₂R; and
   PG² and PG³ are each suitable amino protecting groups,
(e) deprotecting the protected amine moiety of said compound of formula A to form a compound of formula II: 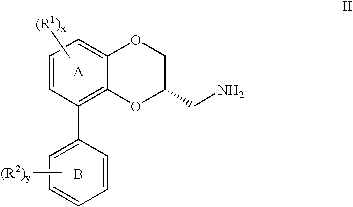
   wherein:
   x is 0-3;
   y is 0-5;
   each R¹ is independently —R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic; and
   each R² is independently R, -Ph, —CN, halogen, —OR, —C(O)NH₂, —C(O)OR, —NHC(O)R, —SO₂R, or —NHSO₂R,
      and
(f) reacting said compound of formula II with suitable acid of formula HX to form a compound of formula II•X.

In certain embodiments, the present invention provides a method for preparing a compound of formula II•HX: 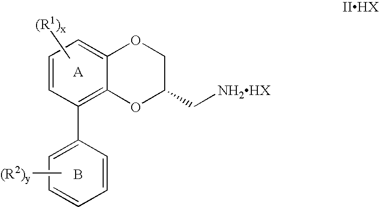
wherein:
x is 0-3;
y is 0-5;
each R³ is independently —R, -Ph, —CN, halogen, —OR, —C(O)NH_{2,}—C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH_{2,}—C(O)OR, —NHC(O)R, —SO₂R, or —NHSO₂R; and
X is the anion of a suitable acid,
   comprising the steps of:
(a) providing a compound of formula J: 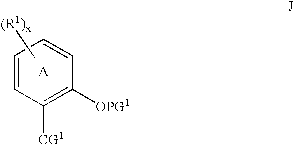
   wherein:
   x is 0-3;
   each R¹ is independently —R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic;
   PG¹ is a suitable hydroxyl protecting group; and
   CG¹ is a coupling group that facilitates transition metal-mediated Cₛₚ₂-Cₛₚ₂ coupling between the attached Cₛₚ₂ carbon and a Cₛₚ₂ carbon bearing a CG² coupling group,
(b) coupling said compound of formula J with a compound of formula H: 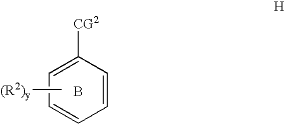
   wherein:
   y is 0-5;
   each R² is independently R, -Ph, —CN, halogen, —OR, —C(O)NH₂, C(O)OR, —NHC(O)R, —SO₂R, or —NHSO₂R;
   each R is independently hydrogen, C₁₋₆ aliphatic or C₁₋₆ fluoroaliphatic; and
   CG² is a coupling group that facilitates transition metal-mediated Cₛₚ₂-Cₛₚ₂ coupling between the attached Cₛₚ₂ carbon and a Cₛₚ₂ carbon bearing a CG¹ coupling group,
      by the action of a suitable transition metal to provide a compound of formula G: 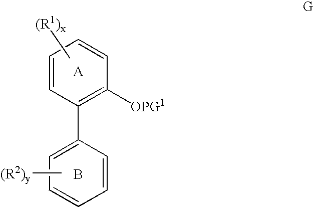
      wherein:
   x is 0-3;
   y is 0-5;
   each R¹ is independently —R, -Ph, —CN, halogen, —R, —(O)NH₂, —C(O)OR, —NHC(O)R, —SO₂R, or —NHSO₂R;
   each R is independently hydrogen, C₁₋₆ aliphatic or C₁₋₆ fluoroaliphatic;
   each R² is independently R, -Ph, —CN, halogen, —OR, —C(O)NH₂, —C(O)OR, —NHC(O)R, —SO₂R, or —NHSO₂R; and
   PG¹ is a suitable hydroxyl protecting group,
(c) introducing a hydroxyl group ortho to the OPG¹ moiety in said compound of formula G to form a compound of formula F: 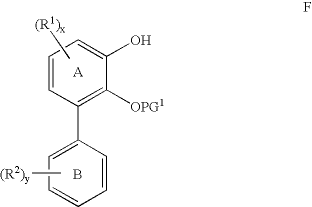
   wherein:
   x is 0-3;
   y is 0-5;
   each R¹ is independently —R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic;
   each R² is independently R, -Ph, —CN, halogen, —OR, —C(O)NH₂, —C(O)OR, —NHC(O)R, —SO₂R, or —NHSO₂R; and
   PG¹ is a suitable hydroxyl protecting group,
(d) glycidating said compound of formula F to form a compound of formula E: 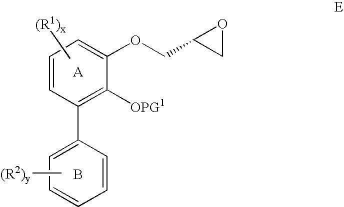
   wherein:
   x is 0-3;
   y is 0-5;
   each R¹ is independently —R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic;
   each R² is independently R, -Ph, —CN, halogen, —OR, —C(O)NH₂, —C(O)OR, —NHC(O)R, —SO₂R, or —NHSO₂R; and
   PG¹ is a suitable hydroxyl protecting group,
(e) opening the distal terminus of the epoxide moiety of said compound of formula E with a suitable nucleophile to afford a compound of formula D: 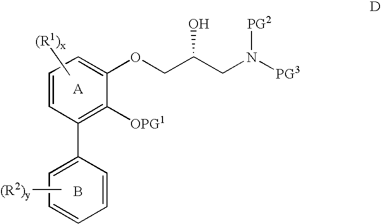
   wherein:
   x is 0-3;
   y is 0-5;
   each R¹ is independently —R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic;
   each R² is independently R, -Ph, —CN, halogen, —OR, —C(O)NH₂, —C(O)OR, —NHC(O)R, —SO₂R, or —NHSO₂R;
   PG¹ is a suitable hydroxyl protecting group; and
   PG² and PG³ are each suitable amino protecting groups,
(f) converting the free hydroxyl moiety of said compound of formula D into a suitable leaving group to afford a compound of formula C: 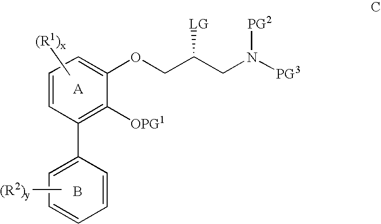
   wherein:
   x is 0-3;
   y is 0-5;
   each R¹ is independently —R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic;
   each R² is independently R, -Ph, —CN, halogen, —OR, —C(O)NH₂, —C(O)OR, —NHC(O)R, —SO₂R, or —NHSO₂R;
   PG¹ is a suitable hydroxyl protecting group;
   PG² and PG³ are each suitable amino protecting groups; and
   LG is a suitable leaving group,
(g) deprotecting the protected hydroxyl moiety of said compound of formula C to form a compound of formula B: 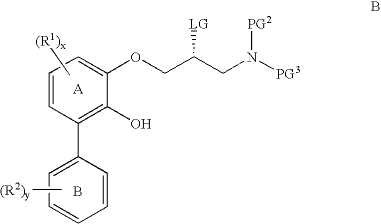
   wherein:
   x is 0-3;
   y is 0-5;
   each R¹ is independently —R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic;
   each R² is independently R, -Ph, —CN, halogen, —OR, —C(O)NH₂, —C(O)OR, —NHC(O)R, —SO₂R, or —NHSO₂R;
   PG² and PG³ are each suitable amino protecting groups; and
   LG is a suitable leaving group,
(h) allowing said compound of formula B to cyclize to form a compound of formula A: 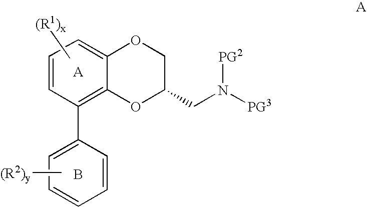
   wherein:
   x is 0-3;
   y is 0-5;
   each R¹ is independently —R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic;
   each R² is independently R, -Ph, —CN, halogen, —OR, —C(O)NH₂, —C(O)OR, —NHC(O)R, —SO₂R, or —NHSO₂R; and
   PG² and PG³ are each suitable amino protecting groups,
(i) deprotecting the protected amine moiety of said compound of formula A to form a compound of formula II: 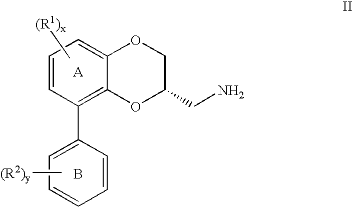
   wherein:
   x is 0-3;
   y is 0-5;
   each R¹ is independently —R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic; and
   each R² is independently R, -Ph, CN, halogen, —OR, —C(O)NH₂, —C(O)OR, —NHC(O)R, —SO₂R, or —NHSO₂R,
      and
(j) reacting said compound of formula II with suitable acid of formula HX to form a compound of formula II•X.

In certain embodiments, the present invention provides a method for preparing a compound of formula A: 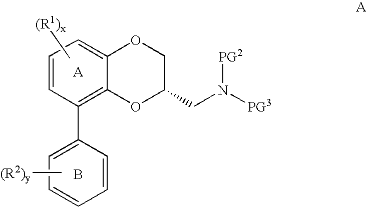
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH_{2,}—C(O)OR, —NHC(O)R, —SO₂R, or —NHSO₂R; and
PG² and PG³ are each suitable amino protecting groups,
   comprising the steps of:
(a) providing a compound of formula Z: 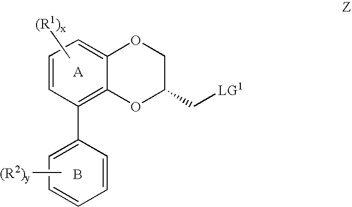
   wherein:
   x is 0-3;
   y is 0-5;
   each R¹ is independently —R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic;
   each R² is independently R, -Ph, —CN, halogen, —OR, —C(O)NH₂, —C(O)OR, —NHC(O)R, —SO₂R, or —NHSO₂R; and
   LG¹ is a suitable leaving group,
      and
(b) treating the compound of formula Z with a suitable amine to afford the compound of formula A.

For compounds of formulae A and Z, each of x, y, R¹, R², PG², PG³, and LG¹ are as defined above and described in embodiments and subembodiments above and herein. In certain embodiments, the compound of formula Z is

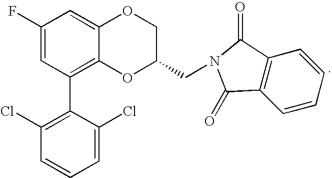

In other embodiments, the present invention provides a method for preparing a compound of formula Z: 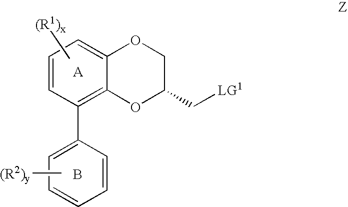
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R; and
LG¹ is a suitable leaving group,
   comprising the steps of:
(a) providing a compound of formula X: 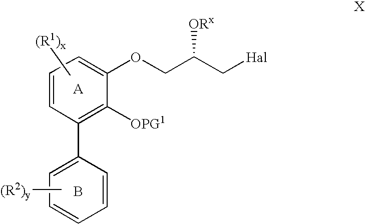
   wherein:
   x is 0-3;
   y is 0-5;
   each R¹ is independently —R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic;
   each R² is independently R, -Ph, —CN, halogen, —OR, —C(O)NH₂, —C(O)OR, —NHC(O)R, —SO₂R, or —NHSO₂R;
   Hal is a halogen; and
   R^{x} is hydrogen or acetyl,
      comprising the steps of:
(b) cyclizing the compound of formula X to form a compound of formula Y: 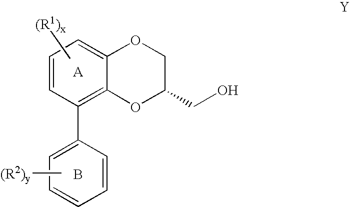
   wherein:
   x is 0-3;
   y is 0-5;
   each R¹ is independently —R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic; and
   each R² is independently R, -Ph, —CN, halogen, —OR, —C(O)NH₂, —C(O)OR, —NHC(O)R, —SO₂R, or —NHSO₂R,
      and
(c) converting the free hydroxyl moiety of the compound of formula Y into a suitable leaving group to afford the compound of formula Z.

For compounds of formulae X, Y, and Z, each of of x, y, R¹, R², R^{x}, hal, and LG¹ are as defined above and described in embodiments and subembodiments above and herein. In certain embodiments, the compound of formula X is 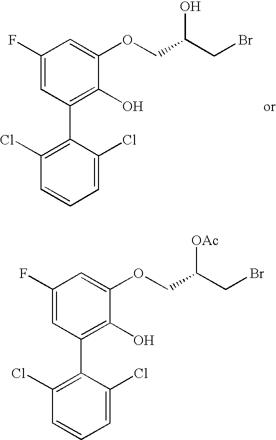
In other embodiments, the compound of formula Y is

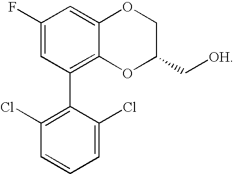

In other embodiments, the present invention provides a method for preparing a compound of formula X: 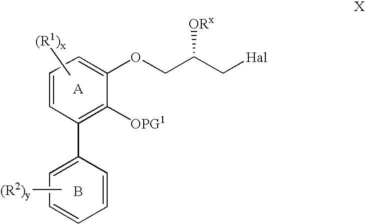
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, C(O)OR, —NHC(O)R, —SO₂R, or —NHSO₂R;
Hal is a halogen; and
R^{x} is hydrogen or acetyl,
   comprising the steps of:
(a) providing a compound of formula E: 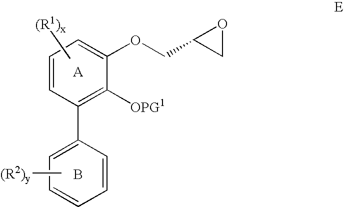
   wherein:
   x is 0-3;
   y is 0-5;
   each R¹ is independently —R, -Ph, —CN, halogen, —OR, —C(O)NH₂, —C(O)OR, —NHC(O)R, —SO₂R, or —NHSO₂R;
   each R is independently hydrogen, C₁₋₆ aliphatic or C₁₋₆ fluoroaliphatic;
   each R² is independently R, -Ph, —CN, halogen, —OR, —C(O)NH₂, —C(O)OR, —NHC(O)R, —SO₂R, or —NHSO₂R; and
   PG¹ is a suitable hydroxyl protecting group,
      and
(b) opening the distal terminus of the epoxide moiety of said compound of formula E with a suitable nucleophile to afford a compound of formula X.

For compounds of formulae X and E, each of of x, y, R¹, R², R^{x}, halogen, and PG¹ are as defined above and described in embodiments and subembodiments above and herein. In certain embodiments, the compound of formula E is

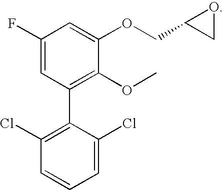

Another aspect of the present invention provides a compound of formula G: 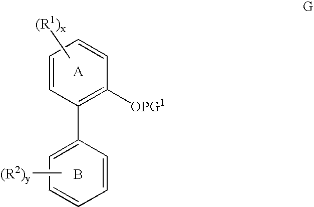
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R; and
PG¹ is a suitable hydroxyl protecting group.

For compounds of formula G, each of x, y, R¹, R², and PG¹ are as defined in embodiments and subembodiments herein. According to one aspect of the present invention, the compound of formula G is

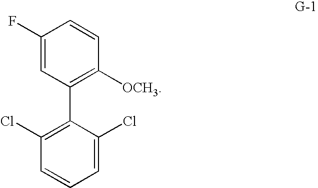

Yet another aspect of the present invention provides a compound of formula F: 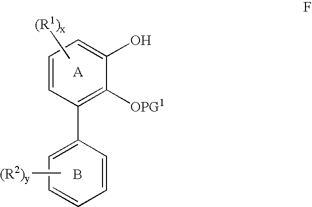
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R; and
PG¹ is a suitable hydroxyl protecting group

For compounds of formula F, each of x, y, R¹, R², and PG¹ are as defined in embodiments and subembodiments herein. According to one aspect of the present invention, the compound of formula F is

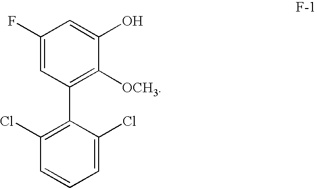

Still another aspect of the present invention provides a compound of formula E: 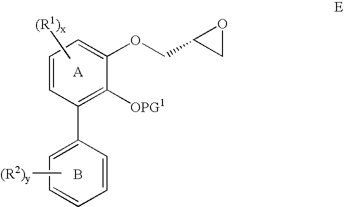
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R; and
PG¹ is a suitable hydroxyl protecting group.

For compounds of formula E, each of x, y, R¹, R², and PG¹ are as defined in embodiments and subembodiments herein. According to one aspect of the present invention, the compound of formula E is

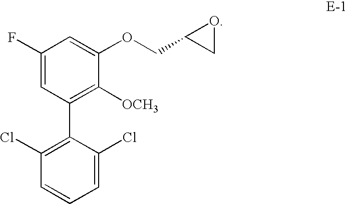

Yet another aspect of the present invention provides a compound of formula D: 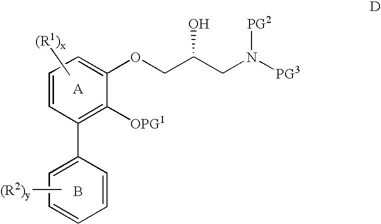
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, c(O)NH₂, —C(O)OR, —NHC(O)R, —SO₂R, or —NHSO₂R;
PG¹ is a suitable hydroxyl protecting group; and
PG² and PG³ are each suitable amino protecting groups.

For compounds of formula D, each of x, y, R¹, R², PG¹, PG², and PG³ are as defined in embodiments and subembodiments herein. According to one aspect of the present invention, the compound of formula D is

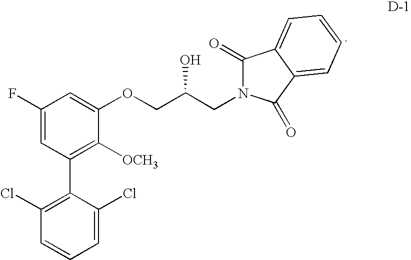

Yet another aspect of the present invention provides a compound of formula C: 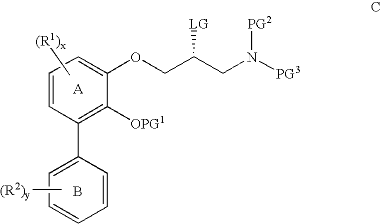
wherein:
x is 0-3;
y is 0-5;
each R¹ is independently —R, -Ph, —CN, halogen, —OR, —C(O)NH₂, C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R;
PG¹ is a suitable hydroxyl protecting group;
PG² and PG³ are each suitable amino protecting groups; and
LG is a suitable leaving group.

For compounds of formula C, each of x, y, R¹, R², PG¹, PG², PG³, and LG are as defined in embodiments and subembodiments herein. According to one aspect of the present invention, the compound of formula C is

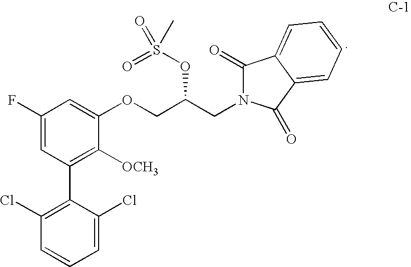

Yet another aspect of the present invention provides a compound of formula B: 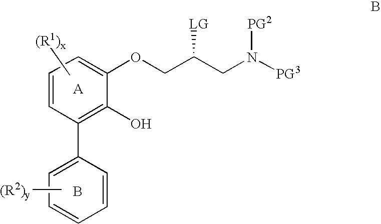
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R;
PG² and PG³ are each suitable amino protecting groups; and
LG is a suitable leaving group.

For compounds of formula B, each of x, y, R¹, R², PG², PG³, and LG are as defined in embodiments and subembodiments herein. According to one aspect of the present invention, the compound of formula B is

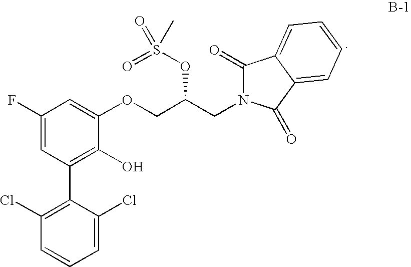

Another aspect of the present invention provides a compound of formula A: 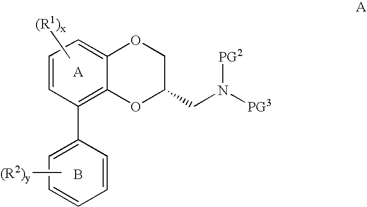
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R; and
PG² and PG³ are each suitable amino protecting groups.

For compounds of formula A, each of x, y, R¹, R², PG², and PG³ are as defined in embodiments and subembodiments herein. According to one aspect of the present invention, the compound of formula B is

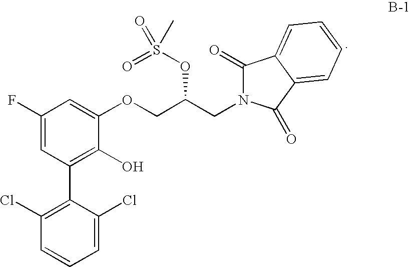

Yet another aspect of the present invention provides a compound of formula II: 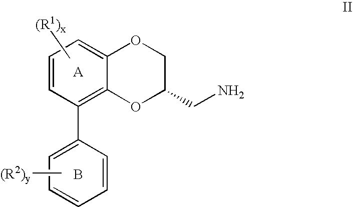
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic; and
each R² is independently R, -Ph, —CN, halogen, —OR, —C(O)NH₂, —C(O)OR, —NHC(O)R, —SO₂R, or —NHSO₂R.

For compounds of formula II, each of x, y, R¹, and R² are as defined in embodiments and subembodiments herein. In certain embodiments, the compound of formula II is selected from those depicted in Table II, above. According to one aspect of the present invention, the compound of formula II is

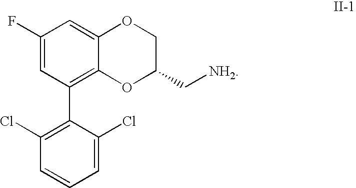

### EXAMPLES

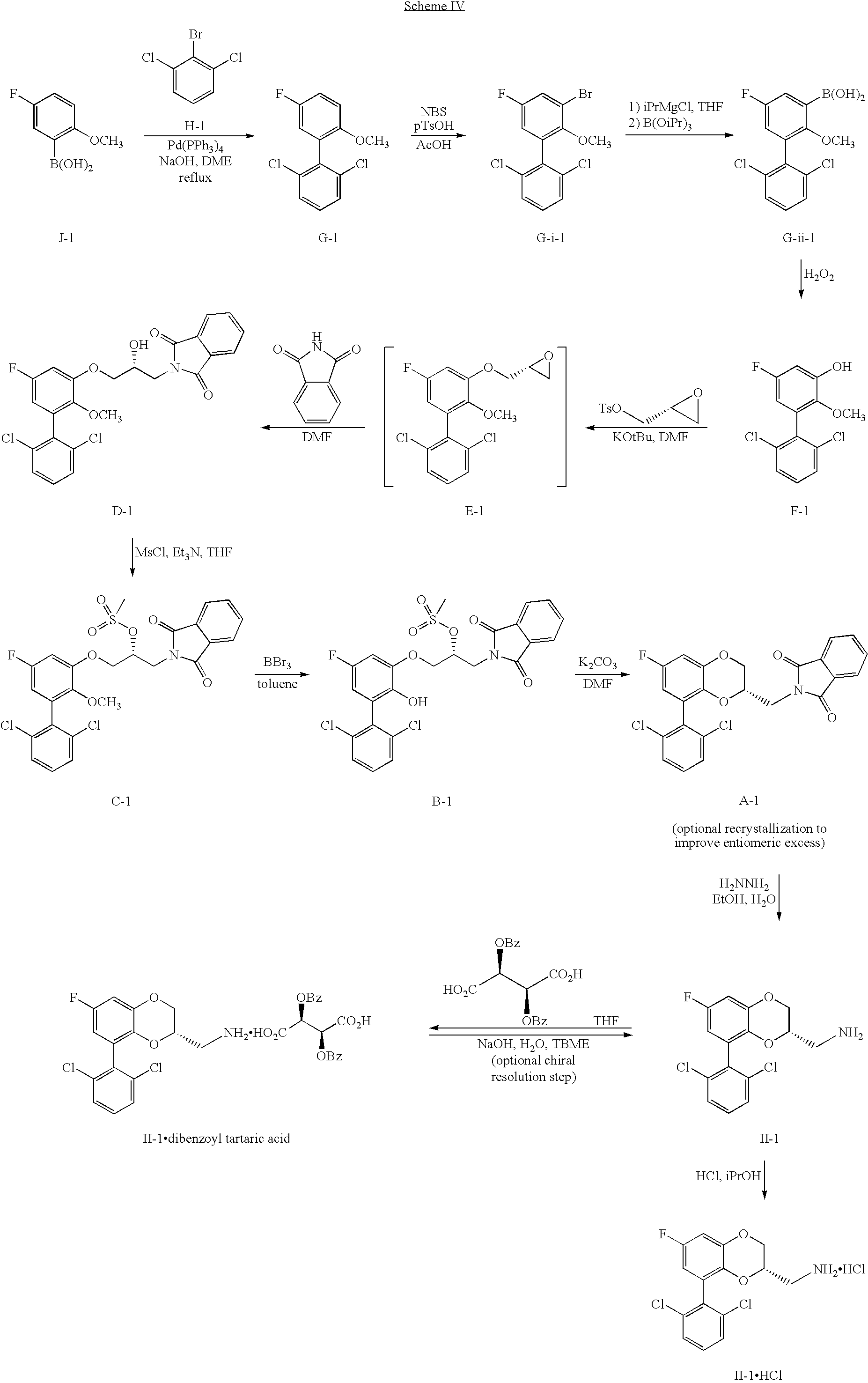

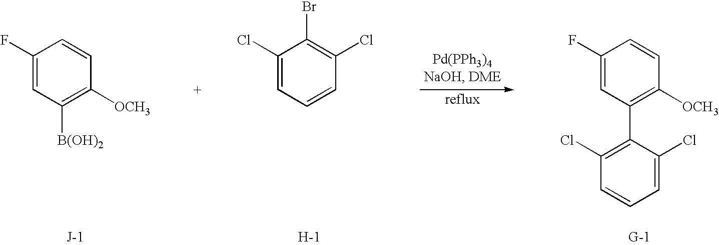

2′,6′-Dichloro-5fluoro-2-methoxy biphenyl (G-1): To a stirring 70° C. solution of 2,6-dichlorobromobenzene, boronic and palladium tetrakis in dimethoxyethane was added an aqueous solution of sodium hydroxide. The mixture was refluxed for 18 hours until less than 1% of starting material was present by HPLC. The mixture was cooled and phases were separated. The reaction mixture was concentrated and heptanes was added. The solution was washed with water. To the product solution was added silica gel. The resulting suspension was stirred for 2 hours then filtered. The intermediate product 2′,6′-dichloro-5-fluoro-2-methoxy-biphenyl in solution in heptanes was concentrated and was used directly for the bromination step. The reaction yield of the Suzuki coupling was 88-92%.

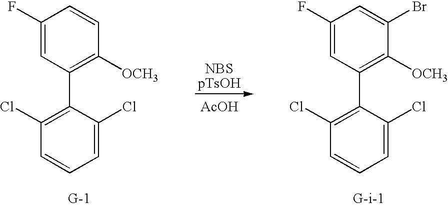

3-Bromo-2′,6′-Dichloro-5 fluoro-2-methoxy Biphenyl (G-i-1): The solution of the intermediate was stripped under vacuum and acetic acid was used for the chase. To the residue was added N-bromosuccinimide, para-toluenesulfonic acid and acetic acid. The suspension was heated to 50-55° C. and stirred for 24 hours. The reaction was quenched with an aqueous solution of sodium metabisulfite and the product was collected by filtration. The yield of the bromination was 88-92%. The overall yield was 77-85%. The crude product was used directly for subsequent step or was recrystallized from acetic acid and water or heptanes.

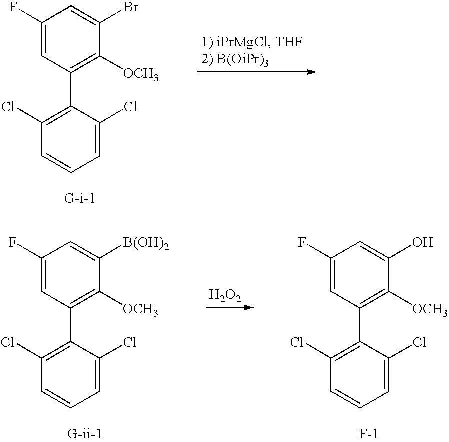

3-Hydroxy-2′,6′-Dichloro-5fluoro-2-methoxy Biphenyl (F-1): A solution of isopropyl magnesium chloride (55 mL, 110 mmol) was added to 3-bromo-2′,6′-Dichloro-5-fluoro-2-methoxy biphenyl (35 g, 100 mmol) dissolved in THF at 0-5° C. Once the reaction was complete (after 4 h), isopropyl borate (28 mL, 120 mmol) was added to the mixture and reacted for a minimum of 4 h. The reaction mixture was quenched with water followed by addition of 30% hydrogen peroxide (16 mL, 150 mmol). After stirring at rt for 12 h, the excess hydrogen peroxide was quenched with Na₂S₂O₅ solution. The organic phase after washing with water was concentrated to dryness to afford the crude product, which was recrystallized from heptanes.

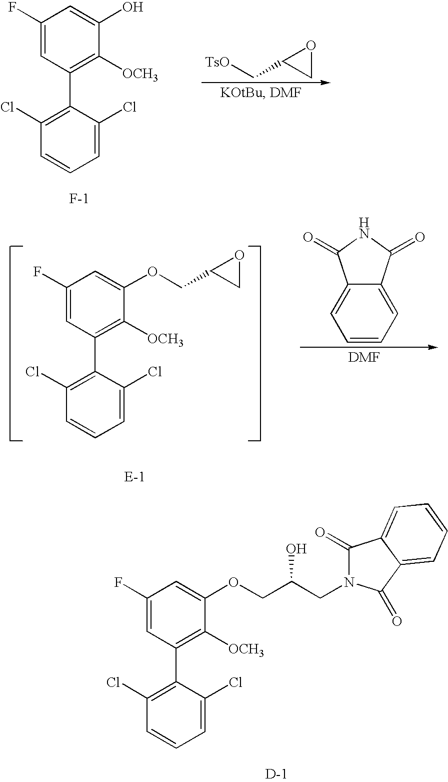

(2R)-3-[3-(2′,6′-Dichlorophenyl)-5-fluoro-2-methoxyphenoxy]-1-N-phthalimidopropan-2-ol (D-1): To a cold solution (0-10° C.) of the 3-hydroxy-2′,6′-dichloro-5-fluoro-2-methoxy biphenyl (150 g, 0.52 mol) in DMF was added potassium tert-butoxide (72 g, 0.63 mol) and the mixture was stirred for 30 min. at 25° C. A solution of R(-)glycidyl tosylate (130 g, 0.57 mol, 99% ee) in DMF was added to the phenolate mixture then heated to 40-50° C. for 1-2 h. When the reaction was completed, phthalimide (76.8 g, 0.52 mol) was added and the mixture was heated to 80° C. for 12 hr. When the reaction was completed, the mixture was cooled to 5° C., isopropyl acetate was added followed by addition of water. The phthalimide intermediate precipitated as a white solid was filtered then dried at 60° C. to give 80-85% yield with 75-90% ee.

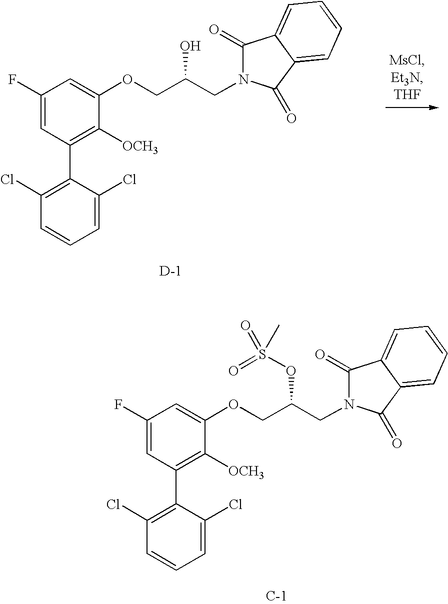

2R-3-[3-(2′,6′-Dichlorophenyl)-5-fluoro-2-methoxyphenoxy]-1-N-phthalimidopropan-2-yl methanesulfonate (C-1): To a stirring solution of 2R-3-[3-(2′,6′-dichlorophenyl)-5-fluoro-2-methoxyphenoxy]-1-N-phthalimidopropan-2-ol (210 g, 0.43 mol) in THF was added triethylamine (89 mL, 0.64 mol) followed by dropwise addition of methanesulfonyl chloride (49 mL, 0.64 mol). The mixture was stirred at 0° C. for 1-2 h until less than 1% of starting material was present by HPLC. After water was added to the mixture at 0° C., the white suspension was stirred at room temperature for 2 h. The product was collected by filtration. The yield of the reaction was 95% with 70-90% ee.

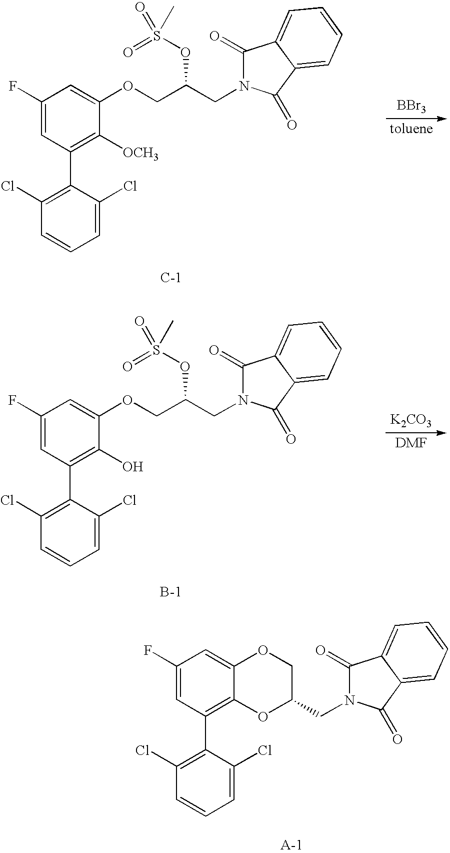

(2S)-2-((8-(2,6-dichlorophenyl)-6-fluoro-2,3-dihydrobenzo[b][1,4]dioxin-2-yl)methyl)isoindoline-1,3Iione (A-1): The 2R-3-[3-(2′,6′-dichlorophenyl)-5-fluoro-2-methoxyphenoxy]-1-N-phthalimidopropan-2-yl methanesulfonate (250 g, 0.44 mol) was suspended in toluene and boron tribromide (176 g, 0.70 mol) was added at 20-25° C. The reaction mixture was stirred for 20 h until the starting material is less than 2%. The reaction was terminated by the addition of water followed by sodium hydroxide 4 N. THF was added to the mixture and the phases were separated. The product solution was concentrated by reduced pressure distillation. Methanol was added for a chase then the intermediate was isolated from methanol by filtration with a yield of 77-84%.

The intermediate was dissolved in DMF and cyclized in presence of potassium carbonate at (20-25° C.) for 20 h. The reaction mixture was filtered then to the filtrates was added water and the product isolated by filtration. The crude product was washed with water then dried. The cyclization yield was 90-92%. The product has a 70-90% ee.

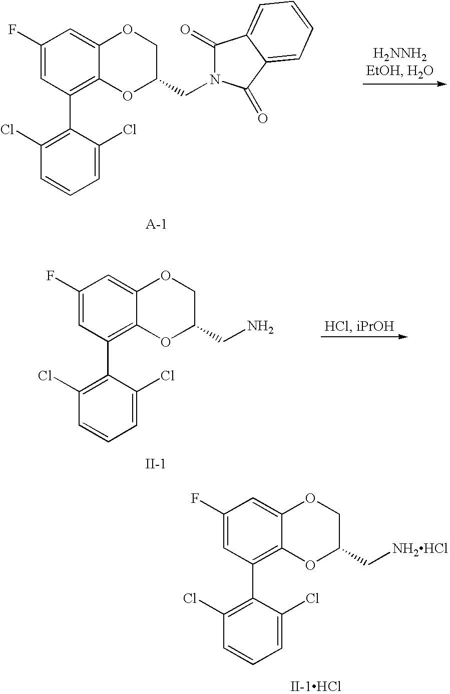

(2S)-(8-(2,6dichlorophenyl)-6-fluoro-2,3-dihydrobenzo [b][1,4]dioxin-2-yl)methanamine hydrochloride (II-1•HCl): To a suspension of (2S)-2-((8-(2,6-dichlorophenyl)-6-fluoro-2,3-dihydrobenzo[b][1,4]dioxin-2-yl)methyl)isoindoline-1,3-dione (213 g) in EtOH-water was added hydrazine (85 mL, 3 eq) dropwise and the mixture was stirred for 2 h at reflux. Water was added and mixture was cooled to 25° C. Worked up with TBME, washed with sodium hydroxide 1N and water. The crude amine in TBME was concentrated under reduced pressure and TBME was replaced by IPA. HCl in IPA (15%) (1 eq) was added at rt. The product was isolated by filtration. Crystallization was repeated as needed to upgrade optical purity of the product.

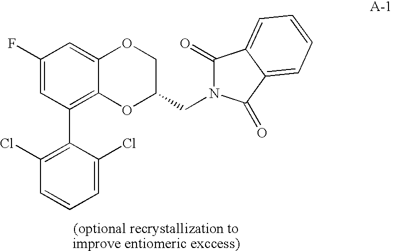

(2S-)-2-((8-(2,6dichlorophenyl)-6-fluoro-2,3-dihydrobenzo[b][1,4]dioxin-2-yl)methyl)isoindoline-1,3-dione (A-1): (2S)-2-((8-(2,6-dichlorophenyl)-6-fluoro-2,3-dihydrobenzo[b][1,4]dioxin-2-yl)methyl)isoindoline-1,3-dione (355 g, 72% ee) was dissolved in toluene at 70-90° C. The mixture was cooled to rt and filtered to remove solid. The filtrate was concentrated to dryness to give product with 60% recovery and 98% ee.

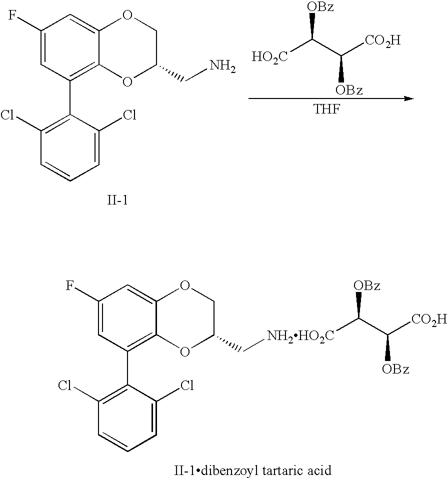

(2S)-(8-(2,6dichlorophenyl)-6-fluoro-2,3-dihydrobenzo[b]1,4]dioxin-2-yl)methanamine Debenzoyl-D-Tartaric acid salt: (2S)-(8-(2,6 dichlorophenyl)-6-fluoro-2,3-dihydrobenzo[b][1,4]dioxin-2-yl)methanamine (10.7 g, 80% ee) solution in THF was added to a solution of dibenzoyl-D-tartaric acid (11.7 g) in THF at reflux. The suspension was cooled to 10-20° C. The product is isolated by filtration. The overall yield is 80-85%. The enantiomeric excess improves to over 99.0%.

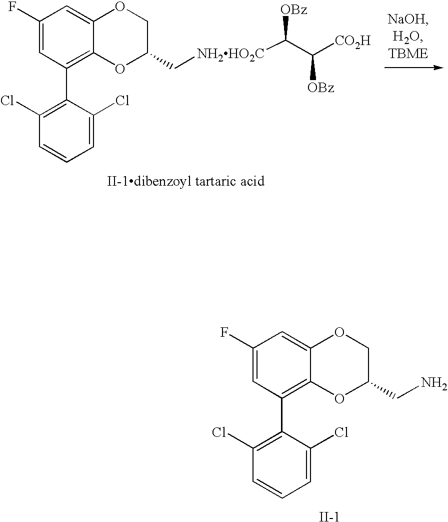

(2S)-(8-(2,6-dichlorophenyl)-6-fluoro-2,3-dihydrobenzo [b][1,4]dioxin-2-yl)methanamine (II-1, 98% ee): To a suspension of (2S)-(8-(2,6-dichlorophenyl)-6-fluoro-2,3-dihydrobenzo[b][1,4]dioxin-2-yl)methanamine dibenzoyl-D-tartaric acid salt (100 g) in TBME/water was added sodium hydroxide solution (30%) (97 g, 4 eq) and the mixture was stirred for 2 h at room temperature. When the reaction was complete, the phase was split and washed with water. The crude amine solution was used directly to the next step.

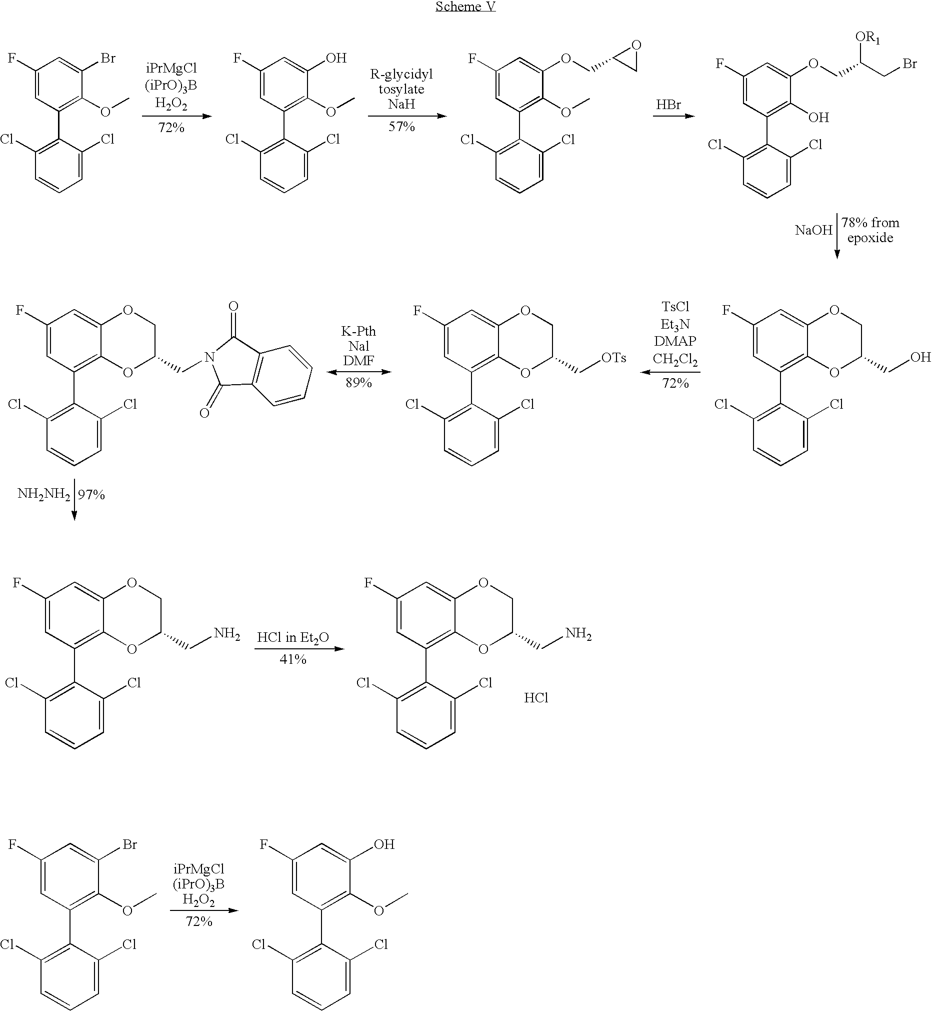

2′,6′-Dichloro-5-fluoro-2-methoxybipheny-3ol: 3-Bromo-2′,6′-dichloro-5-fluoro-2-methoxy-biphenyl (140 g, 400 mmol) was dissolved in THF (500 mL) and cooled to 0 C before adding (220 mL, 440 mmol) of 2N iPrMgCl in THF. The mixture was warmed to RT and stirred for 2 h. The mixture was cooled to 0 C and (112 mL) of (iPrO)₃B was added. Warm to RT and stir for 2 h. Cool the mixture to 0 C and add water (280 mL) followed by (64 mL, ) of 35% hydrogen peroxide. Stir overnight. Add conc HCl (40 mL) and stir until no solids are present. Separate layers and extract the aqueous layer with MTBE. Cool the combined organics in an ice bath and add 250 mL of saturated Na₂S₂O₅ slowly. Separate the layers and wash the organic layer with 2× brine, concentrate, add hexanes and concentrate. Dissolve this residue in hexanes and stir at 0 C for 30 minutes. Collect the solids and air dry to give 49.6 g, 43% yield of the title compound. Second crop 33.8 g, 29% yield.

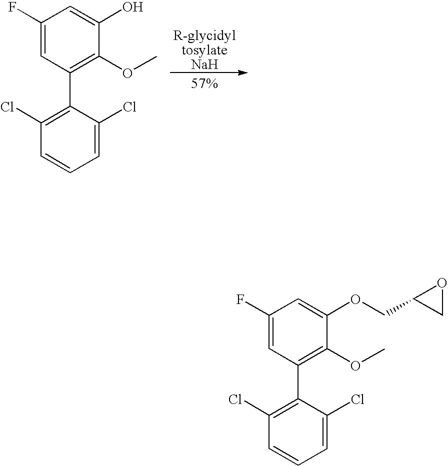

(R)-2-((2′,6′-Dichloro-5-fluoro-2-methoxybiphenyl-3-yloxy)methyl)oxirane: 2′,6′-Dichloro-5-fluoro-2-methoxybiphenyl-3-ol (48 g, 167 mmol) in 300 mL of DMF was treated with 60% NaH (10 g, 252 mmol) keeping the temperature <30 C. After stirring for ½ h a solution of R-glycidyl tosylate (76.2 g, 334 mmol) in DMF was added. Heat to 100 C overnight. The reaction mixture was added to ice water which resulted in gummy solids that stuck to the flask. The liquid was decanted from the flask and extracted twice with methylene chloride. The Ch₂Cl₂ extracts were combined with the gummy solids and washed 2× with brine. Concentration gave ˜90 g of an oil. Column chromatography (30% EtOAc/hexanes) gave 32.95 g, 58% yield of the title compound.

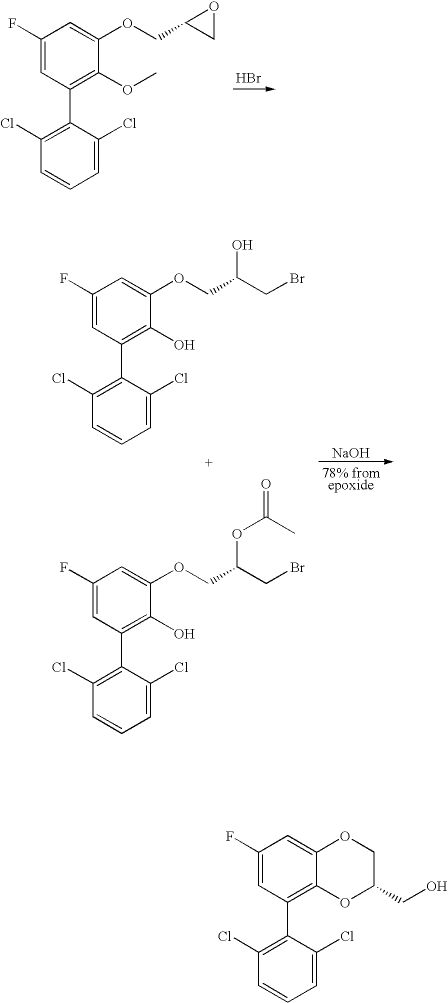

(S)-(8-(2,6Dichlorophenyl)-6-fluoro-2,3-dihydrobenzo[b][1,4]dioxin-2-yl)methanol: (R)-2-((2′,6′-Dichloro-5-fluoro-2-methoxybiphenyl-3-yloxy)methyl)oxirane (32.95 g, 96.0 mmol) was added to 400 mL of 33% HBr in acetic acid and heated to 65° C. for 1 h. Cool to RT and add to 2 L of ice water. This was extracted with CH₂Cl2 twice and the combined organics were washed 2× with water, dried over Na₂SO₄ filtered and concentrated (39.62 g). The concentrate was dissolved in 1 L of methanol and cooled to 0 C before the addition of 500 mL of 2.5 N NaOH. Stir at 0 C for 1.5 h. Add water and extract 3× with CH₂Cl₂. Concentration gave 24.61 g, 78% yield of the title compound.

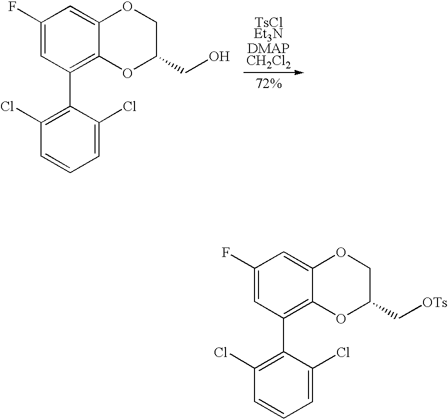

(R)-(8-(2,6-Dichlorophenyl)-6-fluoro-2,3-dihydrobenzo[b][1,4]dioxin-2-yl)methyl 4-methylbenzenesulfonate: (S)-(8-(2,6-Dichlorophenyl)-6-fluoro-2,3-dihydrobenzo[b][1,4]dioxin-2-yl)methanol (24.61 g, 75.0 mmol) was treated with TsCl (17.2 g, 90.0 mmol), iPrNEt (31.4 mL, 180.1 mmol) and cat DMAP in CH₂Cl₂. Stir overnight. TLC (20% EtOAc/hexanes) showed starting material present and another 2 g of TsCl was added. Quenched with dilute HCl and washed with dilute HCl twice followed by water. Concentrated to an oil. Column chromatography (10% EtOAc/hexanes) gave ˜15 g of recovered TsCl. All starting material and product were collected off the column by eluting with EtOAc and concentrating. This mixture was treated with TsCl (17.2 g, 90.0 mmol), iPr2Net (31.4 mL, 180.1 mmol) and 1 g DMAP in CH₂Cl₂. Stirred at RT overnight. Washed with dilute HCl and concentrated. Column chromatography afforded 26.3 g, 72% yield of the title compound.

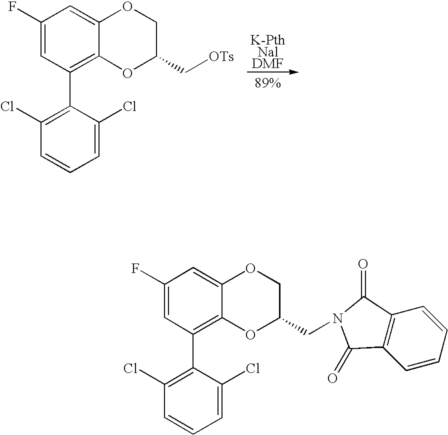

(S)-2-((8-(2,6-Dichlorophenyl)-6-fluoro-2,3-dihydrobenzo[b][1,4]dioxin-2-yl)methyl)isoindoline-1,3dione: (R)-(8-(2,6Dichlorophenyl)-6-fluoro-2,3-dihydrobenzo[b][1,4]dioxin-2-yl)methyl-4-methylbenzenesulfonate (26.1 g, 54.0 mmol), potassium phthalate (21.0 g, 113.4 mmol) and Nal (1 g) in 250 mL of DMF were heated to 85 C for 4 h. Water was added at 55 C and solids gummed out of solution. The liquid was decanted away and the sticky solids were dissolved in EtOAc, washed with water, and concentrated to give 22 g, 89% yield of the title compound.

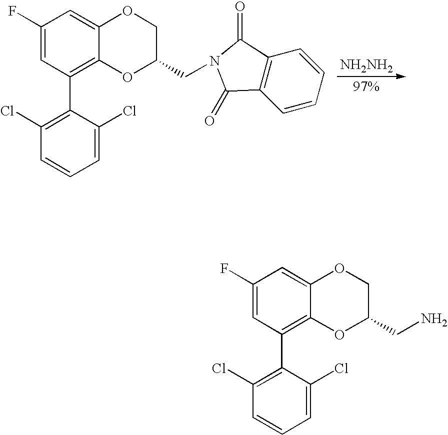

(S)-(8-(2,6 Dichlorophenyl-6-fluoro-2,3-dihydrobenzo[b][1,4]dioxin-2-yl)methanamine: (S)-2-((8-(2,6 Dichlorophenyl)-6-fluoro-2,3-dihydrobenzo[b][1,4]dioxin-2-yl)methyl)isoindoline-1,3-dione (22 g, 48 mmol) in 200 mL of 70% IPA/water was treated with hydrazine hydrate (15 mL) and heated to 85° C. until no starting material was observed by TLC (50% EtOAc/hexanes). 2.5 N NaOH was added and the mixture extracted with MTBE, dried over Na₂SO₄, filtered and concentrated to give 15.25 g, 97% yield of the title compound as an oil.

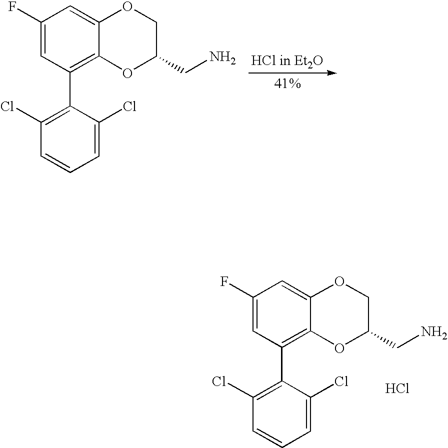

(S)-(8-(2,6-Dichlorophenyl-6-fluoro-2,3-dihydrobenzo[b][1,4]dioxin-2-yl)methanamine Hydrochloride: (S)-(8-(2,6-Dichlorophenyl)-6-fluoro-2,3-dihydrobenzo[b][1,4]dioxin-2-yl)methanamine (30.5 g, 92.9 mmol) in 125 mL of EtOAc was treated with (70 mL, 140 mmol) of 2N HCl in Et₂O. No precipitate was formed. A small aliquot was taken and EtOH was added followed by Et₂O until solids precipitated out of solution. These solids were used as seed crystals. The batch was concentrated and EtOH added followed by Et₂O. The seed crystals were added and the mixture stirred for 1 h. Solids were collected via filtration and washed with Et₂O to afford 14.1 g, 42% yield of the title compound.

In an alternate method, (S)-(8-(2,6-dichlorophenyl)-6-fluoro-2,3-dihydrobenzo[b][1,4]dioxin-2-yl)methanamine (18 g, 54.8 mmol) in 25 mL EtOH/75 mL Et₂O was treated with 50 mL of 2N HCl in ether. Precipitates formed and another 50 mL of ether was added and the mixture stirred at RT overnight. The resulting suspension was cooled in an ice bath for 1 h and the solids collected via filtration to give 8.87 g, 44% yield of the title compound (HPLC area % 100%). The mother liquors were concentrated and Et₂O was added. The mixture was cooled to 0° C. and the solids collected via filtration to give 1.77 g, 8.9% yield of the title compound (HPLC area % 100%). 53% yield overall. Concentrated ML were 94 area % by HPLC.

We claim:

1. A method for preparing a compound of formula A: 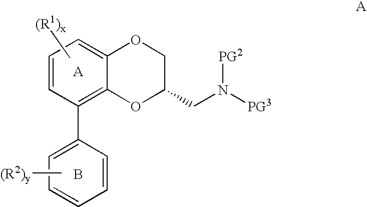
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R; and
PG² and PG³ are each suitable amino protecting groups,
comprising the steps of:
(a) providing a compound of formula B: 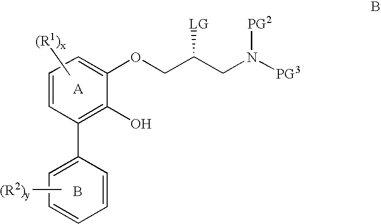
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R;
PG² and PG³ are each suitable amino protecting groups; and
LG is a suitable leaving group,
and
(b) allowing said compound of formula B to cyclize to form a compound of formula A.

2. The method according to claim 1, wherein LG is tosyloxy or mesyloxy.

3. The method according to claim 2, wherein the cyclization of step (b) is performed in the presence of a suitable base.

4. The method according to claim 3, wherein PG² and PG³ form a cyclic imide selected from phthalimide, maleimide, or succinimide.

5. The method according to claim 1, further comprising the steps of:
(a) providing a compound of formula C: 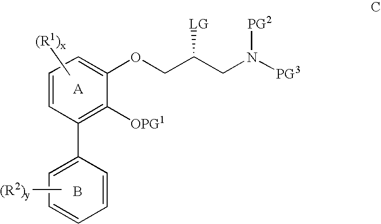
wherein:
x is 0-3;
y is 0-5;
each R¹ is independently —R, -Ph, —CN, halogen, —R, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R;
PG¹ is a suitable hydroxyl protecting group;
PG² and PG³ are each suitable amino protecting groups; and
LG is a suitable leaving group,
and
(b) deprotecting the protected hydroxyl moiety of said compound of formula C to form a compound of formula B.

6. The method according to claim 5, wherein PG¹ is methyl.

7. The method according to claim 6, wherein the deprotection step (b) is performed with BBr₃, iodotrimethylsilane, or a combination of BCl₃ and LiI.

8. The method according to claim 5, further comprising the steps of:
(a) providing a compound of formula D: 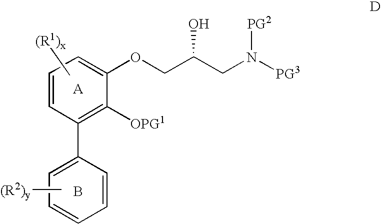
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R;
PG¹ is a suitable hydroxyl protecting group; and
PG² and PG³ are each suitable amino protecting groups,
and
(b) converting the free hydroxyl moiety of said compound of formula D into a suitable leaving group to afford a compound of formula C.

9. The method according to claim 8, further comprising the steps of:
(a) providing a compound of formula E: 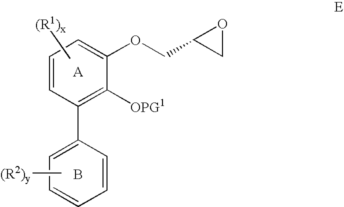
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R; and
PG¹ is a suitable hydroxyl protecting group,
and
(b) treating the compound of formula E with a suitable amine to afford a compound of formula D.

10. The method according to claim 9, wherein the suitable amine is potassium phthalimide.

11. The method according to claim 9, further comprising the steps of:
(a) providing a compound of formula F: 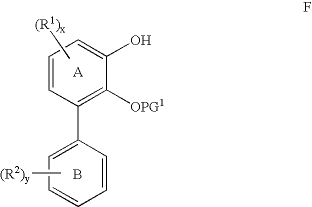
wherein:
x is 0-3;
y is 0-5;
each R¹ is independently —R, -Ph, —CN, halogen, —OR, —C(O)NH₂, C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R; and
PG¹ is a suitable hydroxyl protecting group,
and
(b) glycidating said compound of formula F to form a compound of formula E.

12. The method according to claim 11, wherein step (b) is performed by treating the compound of formula F with a glycidol equivalent selected from epichlorohydrin, epibromohydrin, glycidyl tosylate, glycidyl mesylate, or glycidyl triflate.

13. The method according to claim 12, wherein glycidol equivalent is enantiomerically enriched.

14. The method according to claim 11, further comprising the steps of:
(a) providing a compound of formula G: 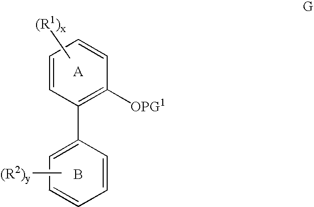
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R; and
PG¹ is a suitable hydroxyl protecting group,
and
(b) introducing a hydroxyl group ortho to the OPG¹ moiety in said compound of formula G to form a compound of formula F.

15. The method according to claim 14, further comprising the steps of:
(a) providing a compound of formula J: 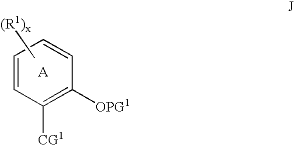
wherein:
x is 0-3;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
PG¹ is a suitable hydroxyl protecting group; and
CG¹ is a coupling group that facilitates transition metal-mediated Cₛₚ₂-Cₛₚ₂ coupling between the attached Cₛₚ₂ carbon and a Cₛₚ₂ carbon bearing a CG² coupling group,
and
(b) coupling said compound of formula J with a compound of formula H: 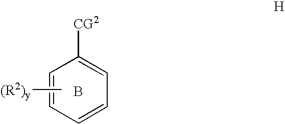
wherein:
y is 0-5;
each R² is independently R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic; and
CG² is a coupling group that facilitates transition metal-mediated Cₛₚ₂-Cₛₚ₂ coupling between the attached Cₛₚ₂ carbon and a Cₛₚ₂ carbon bearing a CG¹ coupling group;
in the presence of a suitable transition metal to form a compound of formula G.

16. The method according to claim 15, wherein said coupling is catalyzed by a palladium species.

17. The method according to claim 16, wherein one of CG¹ or CG² is an electron-withdrawing group selected from Cl, Br, I, or OTf, and the other of CG¹ or CG² is an electropositive group selected from boronic acid, boronic ester, borane, stannane, silyl species, zinc species, aluminum species, magnesium species, or zirconium species.

18. The method according to claim 17, wherein said compound of formula J is

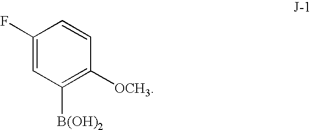

19. The method according to claim 17, wherein said compound of formula H is

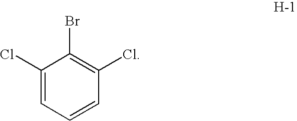

20. A method for preparing a compound of formula A: 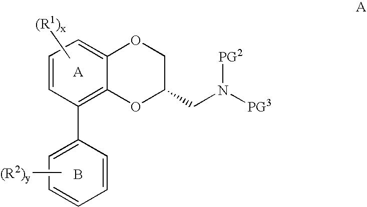
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R; and
PG² and PG³ are each suitable amino protecting groups,
comprising the steps of:
(a) providing a compound of formula Z: 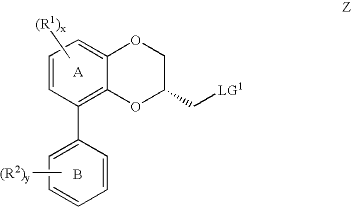
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R; and
LG¹ is a suitable leaving group,
and
(b) treating the compound of formula Z with a suitable amine to afford the compound of formula A.

21. The method according to claim 20, further comprising the step of preparing the compound of formula Z: 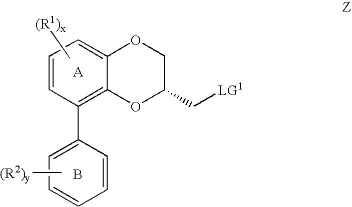
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R; and
LG¹ is a suitable leaving group,
comprising the steps of:
(a) providing a compound of formula X: 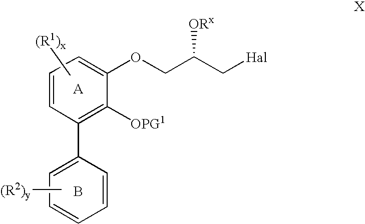
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R;
Hal is a halogen; and
R^{x} is hydrogen or acetyl,
comprising the steps of:
(b) cyclizing the compound of formula X to form a compound of formula Y: 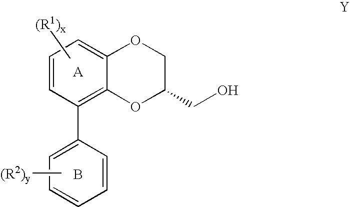
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic; and
each R² is independently R, -Ph, —CN, halogen, —OR, —C(O)NH₂, —C(O)OR, —NHC(O)R, —SO₂R, or —NHSO₂R,
and
(c) converting the free hydroxyl moiety of the compound of formula Y into a suitable leaving group to afford the compound of formula Z.

22. The method according to claim 21, further comprising the step of preparing the compound of formula X: 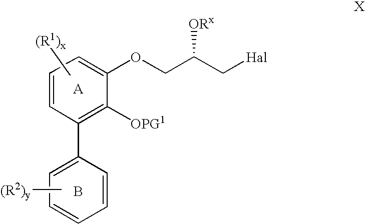
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R;
Hal is a halogen; and
R^{x} is hydrogen or acetyl,
comprising the steps of:
(a) providing a compound of formula E: 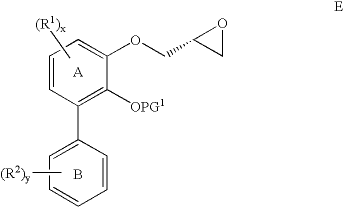
wherein:
x is 0-3;
y is 0-5;
each R¹ is independently —R, -Ph, —CN, halogen, —OR, —C(O)NH₂, —C(O)OR, —NHC(O)R, —SO₂R, or —NHSO₂R;
each R is independently hydrogen, C₁₋₆ aliphatic or C₁₋₆ fluoroaliphatic;
each R² is independently R, -Ph, —CN, halogen, —OR, —C(O)NH₂, —C(O)OR, —NHC(O)R, —SO₂R, or —NHSO₂R; and
PG¹ is a suitable hydroxyl protecting group,
and
(b) opening the distal terminus of the epoxide moiety of said compound of formula E with a suitable nucleophile to afford a compound of formula X.
